(19) United States
(12) Patent Application Publication
(10) Pub. No.: US 2020/0268622 A1
(43) Pub. Date: Aug. 27, 2020

(54) ULTRA-STABLE WATER-IN-OIL HIGH INTERNAL PHASE EMULSIONS FEATURING INTERFACIAL AND BIPHASIC NETWORK STABILIZATION

(71) Applicant: Cornell University, Ithaca, NY (US)

(72) Inventors: Alireza Abbaspourrad, Ithaca, NY (US); Michelle Lee, Ithaca, NY (US)

(21) Appl. No.: 16/800,586

(22) Filed: Feb. 25, 2020

Related U.S. Application Data
(60) Provisional application No. 62/810,437, filed on Feb. 26, 2019.

(51) Int. Cl. A61K 8/06; A61K 8/92; A61K 8/02

(52) U.S. Cl. CPC A61K 8/064; A61K 8/0245; A61K 8/92

(57) ABSTRACT

The present application discloses water-in-oil emulsions comprising an aqueous internal phase and an oleogel external phase, water-in-oil emulsions comprising a hydrogel aqueous internal phase and an oloegel external phase, compositions comprising water-in-oil emulsions, and methods of delivering an incorporated material to a subject by administering compositions comprising water-in-oil emulsions.

This application claims the benefit of U.S. Provisional Patent Application Ser. No. 62/810,437, filed Feb. 26, 2019, which is hereby incorporated by reference in its entirety.

### STATEMENT REGARDING FEDERALLY SPONSORED RESEARCH OR DEVELOPMENT

This invention was made with government support under contract number 1010696 awarded by the USDA National Institute of Food and Agriculture Hatch project and 1719875 awarded by the National Science Foundation. The government has certain rights in the invention.

### FIELD

The present application relates to water-in-oil emulsions, compositions comprising water-in-oil emulsions, and methods of delivering an incorporated material.

### BACKGROUND

High internal phase emulsions (HIPEs) are highly concentrated gelled emulsions with an internal phase volume fraction (Φ) exceeding 0.74 (Cameron et al., “High Internal Phase Emulsions (HIPEs)—Structure, Properties and Use in Polymer Preparation,” Advances in Polymer Science, Vol. 126, Berlin, Heidelberg: Springer, pp. 163-214. (1996)). When the internal phase volume fraction exceeds this value, the dispersed droplets reach their maximum packing density and give rise to highly viscoelastic flow behavior. Due to these viscous flow characteristics, HIPEs have gained popularity for numerous applications, including as templates for porous materials (Hori et al., “Preparation of Porous Polymer Materials Using Water-in-Oil Gel Emulsions as Templates,” Polym. Int. 67(7):909-16 (2018)), foams (Hua et al., “Hydrophilic Polymer Foams with Well-Defined Open-Cell Structure Prepared from Pickering High Internal Phase Emulsions,” J. Polym. Sci. Part A Polym. Chem 51(10):2181-7 (2013)), and cosmetic products (Magdassi, “Delivery Systems in Cosmetics,” Colloids Surfaces A Physicochem. Eng. Asp. 123-4:671-9 (1997)), as solid supports for surface modification (Zhao et al., “Emulsion-Templated Porous Materials (PolyHIPEs) for Selective Ion and Molecular Recognition and Transport: Applications in Electrochemical Sensing,” J. Mater. Chem. 17:(23)2446-53 (2007)) and functional food (Patel et al., “High Internal Phase Emulsion Gels (HIPE-Gels) Prepared Using Food-Grade Components,” RSC Adv. 4(35):18136-40 (2014)), and as scaffolds for tissue engineering (Bokhari et al., “Emulsion-Templated Porous Polymers as Scaffolds for Three Dimensional Cell Culture: Effect of Synthesis Parameters on Scaffold Formation and Homogeneity,” J. Mater. Chem. 17:(38):4088-94 (2007)).

Despite the highly viscoelastic behavior, HIPEs are not kinetically or thermodynamically stable. Typically, the formation of stable HIPEs requires the addition of low molecular weight surfactants (Williams, “High Internal Phase Water-in-Oil Emulsions: Influence of Surfactants and Cosurfactants on Emulsion Stability and Foam Quality,” Langmuir 7(7):1370-7 (1991)) or alternatively the addition of solid colloidal particles in the continuous phase to form Pickering HIPEs (Li et al., “High Internal Phase Emulsions Stabilized Solely by Microgel Particles,” Angew. Chemie—Int. Ed. 48(45):8490-3 (2009)). The selection of surfactant is also critical to form stable HIPEs and often requires large quantities (Sun et al., “Inversion of Particle-Stabilized Emulsions to Form High-Internalphase Emulsions,” Angew. Chemie—Int. Ed. 49(12):2163-6 (2010)). Although Pickering HIPEs have shown increased stability compared with surfactant-stabilized HIPEs, their formation requires chemically-tailored particles with appropriate hydrophobicity, and the resulting emulsion still remains susceptible to phase inversion at high Φ (Aveyard et al., “Emulsions Stabilised Solely by Colloidal Particles,” Adv. Colloid Interface Sci 100-2 (Suppl.):503-46 (2003); and Kralchevsky et al., “On the Thermodynamics of Particle-Stabilized Emulsions: Curvature Effects and Catastrophic Phase Inversion,” Langmuir 21(1):50-63 (2005)).

Water-in-oil (W/O) HIPEs are more difficult to fabricate due to the lack of hydrophobic natural stabilizers, resulting in the limited exploration of these materials (Pang et al., “Water-in-Oil Pickering Emulsions Stabilized by Stearoylated Microcrystalline Cellulose,” J. Colloid Interface Sci. 513:629-37 (2018); Yang et al., “High Internal Phase Emulsions Stabilized by Starch Nanocrystals,” Food Hydrocoll. 82:230-8 (2018); and Liu et al., “Pickering High Internal Phase Emulsions Stabilized by Protein-Covered Cellulose Nanocrystals,” Food Hydrocoll. 82:96-105 (2018)). In addition, the high surface tension of water can also lead to immediate phase inversion during HIPE fabrication when the internal phase volume fraction is high (Welch et al., “Rheology of High Internal Phase Emulsions,” Langmuir 22(4):1544-50 (2006)). Moreover, conventional W/O HIPEs are often stabilized with synthetic surfactants, which can negatively affect the environment and human health (Rebello et al., “Remediation and Green Surfactants,” Environmental Chemistry Letters Springer International Publishing 275-87 (Jun. 20, 2014)). Despite the fact that some more environmentally friendly W/O HIPEs have been developed, they require complicated and time-consuming modification of the stabilizers (e.g., starch or polysaccharide) (Yang et al., “High Internal Phase Emulsions Stabilized by Starch Nanocrystals,” Food Hydrocoll. 82:230-8 (2018); and Liu et al., “Pickering High Internal Phase Emulsions Stabilized by Protein-Covered Cellulose Nanocrystals,” Food Hydrocoll. 82:96-105 (2018)). Therefore, facile methods to form stable and sustainable W/O HIPEs is still in high demand.

In this study, a simple strategy of generating W/O HIPEs upon temperature stimulation to induce the spontaneous formation of Pickering crystals and biphasic networks was proposed. Biodegradable surfactant glycerol monooleate (GMO), a glycerol fatty ester that can solidify and form fat crystals, was utilized to provide spontaneous interfacial Pickering stabilization of the W/O emulsion (Ghosh et al., “Fat Crystals and Water-in-Oil Emulsion Stability,” Curr. Opin. Colloid Interface Sci. 16(5):421-31 (2011); and Milak et al., “Glycerol Monooleate Liquid Crystalline Phases Used in Drug Delivery Systems,” Int. J. Pharm. 478(2):569-87 (2015)). Additionally, structurants were added in both phases, generating carrageenan hydrogel in the internal aqueous phase and beeswax-containing oleogel in the external oil phase. When the network is increased in both phases, or biphasically, the formation of ultra-stable gel-in-gel HIPEs with volume fractions as high as 0.80 was enabled. By tuning the network of each phase, the resultant gel-in-gel HIPEs demonstrate improved stability, avoid phase inversion during fabrication, and can be used as a potential drug co-delivery system. The role of this interfacial and biphasic structuring on HIPE stability was systematically investigated, with the demonstration of protection and release of bioactive compounds for potential application in nutraceutical and biomedical-related fields.

In this work, a gel-in-gel water-in-oil (W/O) high internal phase emulsions (HIPEs) that feature high stability by structuring both phases of the emulsion is presented. Compared to significant advances made in oil-in-water (O/W) HIPEs, W/O HIPEs are extremely unstable and difficult to generate without introducing high concentrations of surfactant. Another main challenge is the low viscosity of both water and oil phases which promotes the instability of W/O HIPEs. Here, ultra-stable W/O HIPEs that feature biphasic structuring were demonstrated, in which hydrogels are dispersed in oleogels, and self-forming, low-concentration interfacial Pickering crystals provide added stability. These W/O HIPEs exhibit high tolerance toward pH shock and destabilizing environments. In addition, this novel ultra-stable gel-in-gel W/O HIPE is sustainable and made solely with natural ingredients without the addition of any synthetic stabilizers. By applying phase structuring within the HIPEs through the addition of various carrageenans and beeswax as structurants, the emulsion's stability and viscoelastic rheological properties can be increased. The performance of these gel-in-gel W/O HIPEs holds promise for a wide range of applications. As a proof-of-concept, herein demonstrated is the application as a gelled delivery system that enables the co-delivery of hydrophilic and hydrophobic materials at maximized loads, demonstrating high resistance to gastrointestinal pHs and a controlled-release profile.

The demand for high-protein food products has rapidly increased in recent years due to the growing health awareness of consumers. Whey protein is an abundant by-product from the dairy industry providing high nutritional value. The high protein and amino acid content of whey protein can become an asset if incorporated in foods. However, astringent taste can be inevitably generated if whey protein is added to food product at low pH and high concentration (>3%) (Sano et al., “Astringency of Bovine Milk Whey Protein,” J. Dairy Sci. 88(7):2312-7 (2005)).

Such astringency is believed to be associated with the interactions between whey protein and salivary component. Upon ingestion of acidified whey protein, complexes are formed through electrostatic interaction between the positively charged whey protein and negatively charged saliva glycoprotein which can then precipitate on the tongue. Such precipitation causes astringency that are collectively described as “puckering” and “drying” sensation (Jobstl et al., “Molecular Model for Astringency Produced by Polyphenol/Protein Interactions,” Biomacromolecules 5(3):942-9 (2004)). Since astringent taste is undesirable to consumers, it is a substantial challenge to mitigate the astringent taste in acidified whey protein food products (Childs et al., “Consumer Perception of Astringency in Clear Acidic Whey Protein Beverages,” J. Food Sci. 75(9):5513-21 (2010)). Current advances in improvement of astringency have mainly focused on modifying protein surface charges (Çelebioğlu et al., “Interactions of Salivary Mucins and Saliva with Food Proteins: a Review,” Crit. Rev. Food Sci. Nutr. 1-20 (2019)), increasing overall food viscosity (Beecher et al., “Factors Regulating Astringency of Whey Protein Beverages,” J. Dairy Sci. 91(7):2553-60 (2008)), and changing food pHs (Vardhanabhuti et al., “Roles of Charge Interactions on Astringency of Whey Proteins at low pH,” J. Dairy Sci. 93(5):1890-9 (2010)). Although some encapsulation methods are developed for targeted delivery of protein (Zhang et al., “Protein Encapsulation in Alginate Hydrogel Beads: Effect of pH on Microgel Stability, Protein Retention and Protein Release,” Food Hydrocolloids 58:308-15 (2016)), very few researchers explore the effect of encapsulation on mitigating whey protein astringency.

High internal phase emulsions (HIPEs) are emulsion containing minimal internal phase volume fraction (Φ) of 0.74 (Cameron et al., “High Internal Phase Emulsions (HIPEs)—Structure, Properties and Use in Polymer Preparation,” Advances in Polymer Science; Vol. 126, Berlin, Heidelberg: Springer pp 163-214 (1996); and Patel et al., “High Internal Phase Emulsion Gels (HIPE-Gels) Prepared Using Food-Grade Components,” RSC Adv. 4(35):18136-40 (2014)). With high fractions of the dispersed droplets, the resultant emulsion become a viscous gel. As descried herein, a novel water-in-oil (W/O) HIPE that can be used to encapsulate hydrophilic nutraceuticals at a high loading content was explored. Therefore, it was hypothesized that this W/O HIPE represents a unique opportunity to simultaneously deliver high whey protein concentration and mitigate the astringent taste in emulsion gel format. In addition, a better health solution can be provided due to high protein loading capacity, low-fat, low-calorie, tunable viscoelastic behavior and high stability in this system. Currently, the aforementioned proteinaceous W/O HIPE systems have not yet been explored in the food industry.

Although multiple advantages are associated with the proteinaceous W/O HIPE, its fabrication remained a great challenge. The difficulty mainly arises from the high surface-activity of whey protein, which could destabilize W/O HIPE with the occurrence of phase inversion, forming oil-in-water (O/W) emulsion eventually. A previous study demonstrated that complexation with polysaccharides can effectively reduce destabilizing effect of whey and increase the emulsion stability (Wagoner et al., “Whey Protein—Pectin Soluble Complexes for Beverage Applications,” Food Hydrocolloids 63:130-8 (2017)).

In this study, first, the functionality of protein-polysaccharide complexes to minimize emulsion destabilizing effects was demonstrated. Such stabilization greatly improved protein loading capacity in the carrier as well as providing great stability for subsequent HIPE. After loading these whey protein-polysaccharide complexes into W/O HIPE, high viscoelasticity and reduced acidified whey protein astringency can be obtained. Finally, the inventors discuss the role of encapsulation on sensory improvements, with in-depth physicochemical characterizations for an effort to provide better health alternatives to human population.

The demand for high-protein food products has rapidly increased in recent years due to the growing health awareness of consumers. Whey protein is an abundant by-product from the dairy industry with high nutritional value. However, it is a substantial challenge to mitigate the astringent taste in acidified whey protein food products. Water-in-oil (W/O) emulsion gel with high internal aqueous phase represents a unique opportunity to simultaneously deliver high whey protein concentration and mitigate the astringent taste in spreadable foods. Whey proteins are prepared and complexed with different polysaccharides at pH 3.5. Formulations based on 75% water phase (containing whey protein and different whey-polysaccharide complexes) and 20% oil phase were manufactured into emulsion gels using a high shear homogenizer. In addition to enhancing the nutritional content, the protein-rich spreadable product can incorporate up to 20 wt % whey in the final product and maintained reasonable particle size (13-30 μm), viscoelasticity (10³-10⁴ Pa), and excellent viscoelastic stability for up to 1 months (25° C.). Incorporation of whey protein at acidic pH in W/O emulsion gel imparts advantages, such as decreased protein degradation as well as increased emulsion spreadability and stability. Furthermore, the resultant products are creamy and less astringent in sensory studies, indicating this product's potential to incorporate acidified whey protein at high concentration and mitigate astringency.

The present application is directed to overcoming the deficiencies in the art.

### SUMMARY

One aspect of the present application is a water-in-oil emulsion comprising an aqueous internal phase, and an oleogel external phase comprising an oleaginous carrier, a surfactant, and an external structurant.

A second aspect of the present application is a water-in-oil emulsion comprising a hydrogel aqueous internal phase comprising an internal structurant, and an oleogel external phase comprising an oleaginous carrier, a surfactant, and an external structurant.

A third aspect of the present application is a composition comprising a water-in-oil emulsion comprising an aqueous internal phase, and an oleogel external phase comprising an oleaginous carrier, a surfactant, and an external structurant, and one or more incorporated materials.

A fourth aspect of the present application is a composition comprising a water-in-oil emulsion comprising a hydrogel aqueous internal phase comprising an internal structurant, and an oleogel external phase comprising an oleaginous carrier, a surfactant, and an external structurant, and one or more incorporated materials.

Another aspect of the present application is a method of delivering an incorporated material to a subject comprising selecting a subject in need of the incorporated material and administering, to the selected subject, a composition of the present application.

The combination of GMO (i.e. glycerol monooleate) interfacial droplet stabilization and the structured network provided by beeswax externally and carrageenan internally enables the fabrication of W/O HIPEs with excellent stability. The fabrication process utilizes renewable materials and provides a robust technique for overcoming the difficulties in forming ultra-stable W/O HIPEs. These gel-in-gel HIPEs can provide insight into promising applications, such as pH-responsive release for hydrophilic and hydrophobic nutraceuticals, with high environmental stability. By creating an emulsion with biphasic structures, the rheological behavior of current conventional W/O HIPEs is enhanced. In addition, this method requires very low amounts of surfactants (0.25 wt %) and structurants (0.75 wt %) in the total system, as well as providing high drug loading capacity. Compared to conventional polyHIPEs and Pickering HIPEs, this method exhibits great potential in terms of efficiency, rheological performance, encapsulation capacity, and stability. These novel gel-in-gel HIPEs, fabricated with solely natural materials, may prove valuable for the biological, chemical, food, and pharmaceutical industries.

This work also presents a promising way of mitigating whey protein isolate (WPI) astringency in a spreadable product by incorporating it into a W/O HIPE system. The inventors tailored WPI into protein-polysaccharide complexes (PPCs) that do not destabilize HIPE emulsion and can enable high protein loading capacity (20 w %). By incorporating WPI as PPC, the resulting HIPE showed higher viscoelastic behavior suggesting higher stability and minimized interaction with oral mucin, suggesting lower astringency. Results presented here suggested that the electrostatic interactions between mucin and WPI play an important role in astringency development. Through reducing WPI interactions with mucin by modifying surface charge or physically creating an oil barrier around it, WPI astringency can be greatly mitigated. In addition, the inventors' method provides insight into protein encapsulation techniques, which can be used for numerous promising applications. This novel high protein spreadable product, generated with simple fabrication techniques showed its promising value for biological, food, and pharmaceutical industries.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole. In addition, preferences and options for a given aspect, feature, embodiment, or parameter of the invention should, unless the context indicates otherwise, be regarded as having been disclosed in combination with any and all preferences and options for all other aspects, features, embodiments, and parameters of the present application.

### BRIEF DESCRIPTION OF THE DRAWINGS

FIG. 1 shows a schematic illustration of the fabrication of W/O HIPEs with different phase structuring.

FIGS. 2A-2F are optical (FIGS. 2A-2C) and CLSM (FIGS. 2D-2F) images of the HIPE microstructures stabilized by 1 wt % GMO (FIGS. 2A and 2D), 3 wt % beeswax (FIGS. 2B and 2E), and both 1 wt % GMO and 3 wt % beeswax (FIGS. 2C and 2F) in the external phase at Φ=0.75. FITC is incorporated as the only water-soluble indicator.

FIGS. 3A-3C depict stability of HIPEs made from only 1 wt % GMO (FIG. 3A), only 3 wt % beeswax (FIG. 3B), and 1 wt % GMO and 3 wt % beeswax (FIG. 3C) in the oil phase stored at 25° C. for one day.

FIGS. 4A-4C depicts O-HIPEs featuring different internal phase volume fractions. FIGS. 4A-4B show images (FIG. 4A) and rheological measurements (FIG. 4B) of O-HIPEs prepared at different internal phase volume fractions. FIG. 4C shows the microstructure of different internal phase volume fraction O-HIPEs as shown by (top row) optical and (bottom row) CLSM microscopy.

FIG. 5 shows the particle size distribution of O-HIPEs made with 1 wt % GMO and 3 wt % beeswax in the external phase and internal volume fractions of 0.70, 0.75, 0.80, and 0.85.

FIG. 6 depicts the stability of O-HIPEs made with 1 wt % GMO and 3 wt % beeswax in the external phase at internal volume fractions of 0.80 (left) and 0.85 (right). The samples were stored for 2 days at 25° C.

FIGS. 7A-7D depict fabrication of O-HIPEs at Φ=0.75 using different beeswax concentrations in the external phase. FIG. 7A is an image of the O-HIPEs formed at different wax concentrations in the external phase. FIG. 7B depicts the microstructure of the O-HIPEs shown via CLSM imaging. FITC is incorporated as the only water-soluble indicator. FIG. 7C shows contact angle measurements of water on oleogel films of 1 wt % GMO and different beeswax concentrations (0.5-10 wt %). FIG. 7D shows rheological measurements of the O-HIPEs structured with different wax concentrations.

FIG. 8 depicts the interfacial tension of water or 1 wt % carrageenans (internal phase) dispensed in different external phases. The interfacial tension measurements were conducted in a temperature-controlled chamber at 50° C. All measurements are expressed in the unit of mN/m.

FIG. 9 depicts the interfacial tension of water (internal phase) dispensed in oil external phases composed of 1 wt % GMO and different beeswax concentration (0.5-10 wt %). The interfacial tension measurements were conducted in a temperature-controlled chamber at 50° C. All measurements are expressed in the unit of mN/m.

FIGS. 10A-10B show visual images (FIG. 10A) and rheological measurement (FIG. 10B) of bulk oleogel made with beeswax at different weight concentrations.

FIG. 11 shows the particle size distribution of O-HIPEs made with 1 wt % GMO and beeswax concentrations of 0.5, 1, 3, 5, and 10 wt % in the external phase.

FIG. 12 shows the stability of the O-HIPE made with 1 wt % GMO and 10 wt % beeswax in the oil phase at an internal volume fraction of 0.75. The sample was stored for 2 days at 25° C.

FIGS. 13A-13D depict fabrication of HIPEs at Φ=0.8 with 3 wt % beeswax and 1 wt % GMO and different types of carrageenans in the internal phase at 1 wt %. FIG. 13A shows Images of the gel-in-gel HIPEs. FIG. 13B shows the microstructure of these HIPEs made with (a) κ-carrageenan, (b) ι-carrageenan, and (c) λ-carrageenan in the internal phase, as shown by CLSM (in the left three columns, aqueous and oil stains) and optical microscopy (right column). FIG. 13C shows rheological measurements of the HIPEs that were internally-structured with different types of carrageenans. FIG. 13D shows contact angle measurements of the different types of carrageenan on the oleogel film consisting of 3 wt % beeswax and 1 wt % GMO.

FIGS. 14A-14C depict gel-in-gel HIPEs featuring different ι-carrageenan concentrations. FIGS. 14A-14B show images (FIG. 14A) and rheological measurements (FIG. 14B) of gel-in-gel HIPEs prepared with different ι-carrageenan concentrations in the internal phase. FIG. 14C is the optical microscopy showing microstructure of gel-in-gel HIPEs with different ι-carrageenan concentrations.

FIG. 15 shows the particle size distribution of gel-in-gel HIPEs made with various types of carrageenans (κ, ι, λ) in the internal phase at 1 wt % and 1 wt % GMO and 3 wt % beeswax in the external phase.

FIGS. 16A-16D depict cryo-SEM images (top rows) and EDS (bottom rows) measurements of the O-HIPE (FIG. 16A) and gel-in-gel HIPEs (FIGS. 16B-16D) made with κ-carrageenan, (FIG. 16B) ι-carrageenan (FIG. 16C), and λ-carrageenan (FIG. 16D).

FIGS. 17A-17D are images of the O-HIPE (FIG. 17A) and gel-in-gel HIPEs (FIGS. 17B-17D) made from κ-carrageenan (FIG. 17B), λ-carrageenan (FIG. 17C), and ι-carrageenan (FIG. 17D) stored over time at 25° C. in open atmosphere.

FIG. 18 shows images of the O-HIPE and gel-in-gel HIPEs made from κ-carrageenan, ι-carrageenan, and λ-carrageenan stored over time under vacuumed conditions.

FIG. 19 shows images of the O-HIPE and gel-in-gel HIPEs made from κ-carrageenan, ι-carrageenan, and λ-carrageenan stored over time under 37° C.

FIGS. 20A-20E depict incorporation of anthocyanin and β-carotene into O-HIPE and gel-in-gel HIPEs. FIG. 20A shows images of anthocyanin-incorporated (top) and anthocyanin-β-carotene incorporated (bottom) O-HIPE and gel-in-gel HIPEs. FIGS. 20B-20C show the release of anthocyanin at 37° C. from O-HIPE and gel-in-gel HIPEs at pH 1 (FIG. 20B) and pH 5 (FIG. 20C). FIGS. 20D-20E show the release of β-carotene at pH 1 (FIG. 20D) and pH 5 (FIG. 20E).

FIG. 21A demonstrates the stability of an anthocyanin-incorporated ι-carrageenan gel-in-gel HIPE at different pH and 25° C. FIG. 21B demonstrates the stability of β-carotene and anthocyanin co-encapsulated in the gel-in-gel HIPE after extraction using n-Hexanes (FIG. 21B).

FIGS. 22A-22C depict O-HIPEs featuring different GMO concentrations in the external phase. FIGS. 22A-22B show images (FIG. 22A) and rheological measurements (FIG. 22B) of O-HIPEs prepared with different GMO concentrations. FIG. 22C shows the optical microscopy showing microstructure of O-HIPEs with different GMO concentrations.

FIG. 23 shows the particle size distribution of O-HIPEs made with 3 wt % beeswax and different GMO concentrations (0.5-3%) in the external phase at internal volume fractions of 0.75.

FIGS. 24A-24C depict gel-in-gel (t-carrageenan) HIPEs featuring different GMO concentrations. FIGS. 24A-24B show images (FIG. 24A) and rheological measurements (FIG. 24B) of gel-in-gel (t-carrageenan) HIPEs prepared with different GMO concentrations. FIG. 24C is the optical microscopy showing microstructure of gel-in-gel (t-carrageenan) HIPEs with different GMO concentrations.

FIG. 25 shows three phase contact angle of water on 3% beeswax oleogel film immersed in either canola oil or canola oil-GMO mixture.

FIG. 26 shows the mechanism by which the protein-polysaccharide complex (PPC), and whey protein isolate (WPI) loaded HIPEs can reduce protein astringency perception under acidic condition.

FIGS. 27A-27D depict fabrication of HIPEs loaded with different WPI concentrations at pH 3.5 in the internal phase. FIG. 27A shows image of HIPEs loaded with different WPI concentrations in the internal phase. FIG. 27B shows CLSM imaging of HIPEs' microstructure. FIG. 27C shows rheological measurements of the HIPEs loaded with different WPI concentrations, and FIG. 27D shows particle sizes of the internal droplets in HIPEs.

FIG. 28 shows particle size distribution of HIPEs made with different WPI concentrations in the aqueous phase.

FIGS. 29A-29C depict HIPEs featuring 1% WPI in the internal phase with different internal phase volume fractions. FIG. 29A is an image of HIPEs prepared at different internal phase volume fractions. FIG. 29B shows the corresponding microstructure of HIPEs observed from confocal laser scanning microscopy. FIG. 29C shows rheological measurements of HIPEs at different internal phase volume fractions.

FIG. 30 shows particle size distribution of HIPEs (1% WPI) internal volume fractions of 0.65, 0.70, 0.75, and 0.80.

FIGS. 31A-31D depict characterization of WPI-carrageenan PPC. FIG. 31A shows the visual appearance of the κ-, ι-, λ-PPC. FIG. 31B shows SEM of the κ-, ι-, λ-PPC. FIG. 31C shows particle size measurements of WPI and PPCs (κ-, ι-, λ-) before and after mixing with 0.1 w % mucin. FIG. 31D shows potential of WPI and PPCs (κ-, ι-, λ-) before and after mixing with 0.1 w % mucin.

FIG. 32 shows turbidity of the control (0.1% WPI), PPC solutions (0.1%, κ-, ι-, λ-) and the corresponding mixture with 0.1% mucin.

FIGS. 33A-33C depict real-time QCM-D characterizations of interactions between WPI and mucin. FIG. 33A shows a demonstration of the coating mechanisms of the WPI and κ-, ι-, λ-PPC on the QCM-D sensors. FIG. 33B is QCM-D frequency output demonstrating WPI-mucin interactions (control). FIG. 33C shows the amount of WPI-carrageenans (κ-, ι-, λ-, control) and PPCs (κ-, ι-, λ-) attached onto mucin-coated QCM-D gold sensors.

FIGS. 34A-34C depict HIPEs. FIG. 34A shows the visual appearance of HIPEs incorporated with κ-, ι-, λ-PPCs. FIG. 34B shows rheological measurement of HIPEs loaded with 20% protein. FIG. 34C shows CLSM of HIPEs loaded with κ-, ι-, λ-PPC. Here, HIPEs were formulated with PPC containing 20% WPI.

FIG. 35 shows the particle size distribution of HIPEs (Φ=0.75) incorporating 20% κ-, ι-, λ-PPC.

FIG. 36 shows the astringency sensory score of PPC (κ-, ι-, λ-) and the corresponding HIPEs formulated containing 10% WPI (n=15). The dash line indicates the score of control (10% WPI solution).

FIGS. 37A-37C shows that a probiotic loaded HIPE can maintain probiotic viability. Probiotics were stored at 25° C. for 2 weeks (14 days) and tested for viability at 7 day intervals. The probiotic viability was characterized through plating probiotic loaded HIPEs on agar growth medium in intervals of 7 days. FIG. 37A shows growth at day 1. FIG. 37B shows growth at day 7. FIG. 37C shows growth at day 14.

FIG. 38A shows loading of milk protein concentration (MPC) into the internal phase of W/O HIPEs. FIG. 38B shows O-HIPEs made using (from left to right) milk fat as external phase, milk as internal phase, and chocolate milk as internal phase. The milk fat is used as an oleogel as it contains natural saturated fat (structurant) and liquid oil (oleaginous carrier) in the O-HIPE.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

As used in this application, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

Where a range of values is provided, it is intended that each intervening value between the upper and lower limit of that range and any other stated or intervening value in that stated range is encompassed within the disclosure. For example, if a range of 1 to 10 minutes is stated, it is intended that 2 minutes, 3 minutes, 4 minutes, 5 minutes, 6 minutes, 7 minutes, 8 minutes, and 9 minutes are also explicitly disclosed, as well as the range of values greater than or equal to 1 minute and the range of values less than or equal to 10 minutes.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual component of embodiments of the present application, or groups thereof, or any members of a claimed group, less than the full measure of this disclosure can be claimed for any reason.

One aspect of the present application is a water-in-oil emulsion comprising an aqueous internal phase, and an oleogel external phase comprising an oleaginous carrier, a surfactant, and an external structurant.

Another aspect of the present application is a water-in-oil emulsion comprising a hydrogel aqueous internal phase comprising an internal structurant, and an oleogel external phase comprising an oleaginous carrier, a surfactant, and an external structurant.

As used herein, an “emulsion” is a fluidic state which exists when a first fluid is dispersed in the form of droplets in a second fluid that is typically immiscible or substantially immiscible with the first fluid. Examples of common emulsions are oil in water (o/w) and water in oil (w/o) emulsions.

Emulsions can be characterized as having internal and external phases, where droplets of the internal phase are formed within the external phase. In water-in-oil emulsions, the internal phase is water based and the external phase is oil based.

As such, the term “aqueous internal phase,” as used herein, refers to the water based droplets formed in the emulsion.

The term “oleogel external phase,” as used herein, refers to an oil based external phase structured with an external structurant to form an oleogel.

As used herein, the term “oleaginous carrier” refers to the oil on which the oleogel external phase is based. Oleagious carriers may be derived, for example, from a plant source, such as a vegetable, a nut, a fruit, etc., or from an animal source, such as a fish.

In an embodiment, the oleaginous carrier may be almond oil, apricot kernel oil, argan oil, avocado oil, baobab oil, camelina oil, canola oil, carrot oil, castor oil, chile oil, citronella oil, corn oil, cottonseed oil, cranberry seed oil, flax seed oil, grapeseed oil, hazelnut oil, hemp seed oil, jojoba oil, macadamia nut oil, meadowfoam seed oil, mustard oil, oat emollient, olive oil, peanut oil, pine seed oil, poppy seed oil, rapeseed oil, red raspberry seed oil, rice bran oil, rose hip oil, safflower oil, sesame oil, sesame seed oil, soybean oil, sunflower oil, tea oil, truffle oil, walnut oil, wheat germ oil, fish oil, or combinations thereof.

In an embodiment, the oleaginous carrier may be fish oil derived from anchovies, carp, catfish, cod, flounder, gemfish, grouper, halibut, herring, jack, kippers, mackerel, mahi mahi, orange roughy, pilchards, Pollock, salmon, sardines, snapper, sprats, swordfish, tilefish, trout, tuna, whitebait, or combinations thereof.

In another embodiment, the oleaginous carrier is derived from eel, oyster, prawn, shark, or combinations thereof.

In one embodiment, the oleaginous carrier comprises omega-3 fatty acids (e.g. eicosapentaenoic acid (EPA) and/or docosahexaenoic acid (DHA)).

In one embodiment, the oleaginous carrier is canola oil.

In an embodiment, the oleogel external phase comprises at least about 90 wt %, 80 wt %, 70 wt %, 60 wt %, 50 wt %, 40 wt %, 30 wt %, 20 wt %, or 10 wt % of the oleaginous carrier.

As used herein, the term “surfactant” refers to a substance which tends to reduce the surface tension of a liquid in which it is dissolved.

The surfactant(s) of the present application are not limited by this disclosure. Generally, surfactants suitable for inclusion in the oleogel external phase are soluble or dispersable in the oleaginous carrier.

In an embodiment, the surfactant is a nonionic surfactant. Examples of nonionic surfactants may include ethoxylated linear alcohols, ethoxylated alkyl phenols, ethoxylated thiols, acid ethoxylated fatty acids, glycerol esters, esters of hexitols and cyclic anhydrohexitols, amine and amide derivatives, alkylpolyglucosides, ethleneoxide/propyleneoxide copolymers, polyalcohols and ethyoxylated polyalcohols, thiols (mercaptans) and derivatives, and combinations thereof.

In an embodiment, the surfactant is a glycerol ester. In another embodiment, the surfactant is glycerol monooleate.

In an embodiment, the surfactant is present in an amount of from about 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt % of the oleaginous carrier.

Nonionic surfactants can be characterized by their hydrophilic-lipophilic balance (HLB) (Schott, “Hydrophilic-Lipophilic Balance, Solubility Parameter, and Oil-Water Partition Coefficient a Universal Parameters of Nonionic Surfactants,” J. Pharm. Sci. 84(10):1215-22 (1995), which is hereby incorporated by reference in its entirety), which is a measure of the degree to which it is hydrophilic or lipophilic. In embodiments, the surfactants has a HLB of less than about 10. In another embodiment, the surfactant has a HLB of between about 3 and about 6. In yet another embodiment, the surfactant has a HLB of between about 7 to about 9.

As used herein, the term “external structurant” is any material that is added to the oleogel external phase to provide rheological and stability benefits.

In an embodiment, the external structurant is a saturated fat. In another embodiment, the external structurant is solid at room temperature. The external structurant may be of plant origin, animal origin, synthetic origin, or combinations thereof.

In an embodiment, the external structurant may be cocoa butter, coconut oil, margarine, palm kernel oil, palm oil, beef fat, beeswax, butter, chicken fat, ghee, milk fat, pork fat, hydrogenated oils, partially hydrogenated oils, or combinations thereof.

In one embodiment, the external structurant is beeswax.

In embodiments, the external structurant is present in an amount of about from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 4 to 5, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 5 to 6, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 20, 9 to 19, 9 to 18, 9 to 17, 9 to 18, 9 to 17, 9 to 16, 9 to 15, 9 to 14, 9 to 13, 9 to 12, 9 to 11, 9 to 10, 10 to 20, 10 to 19, 10 to 18, 10 to 17, 10 to 16, 10 to 15, 10 to 14, 10 to 13, 10 to 12, 10 to 11, 11 to 20, 11 to 19, 11 to 18, 11 to 17, 11 to 16, 11 to 15, 11 to 14, 11 to 13, 11 to 12, 12 to 20, 12 to 19, 12 to 18, 12 to 17, 12 to 16, 12 to 15, 12 to 14, 12 to 13, 13 to 20, 13 to 19, 13 to 18, 13 to 17, 13 to 16, 13 to 15, 13 to 14, 14 to 20, 14 to 19, 14 to 18, 14 to 17, 14 to 16, 14 to 15, 15 to 20, 15 to 19, 15 to 18, 15 to 17, 15 to 16, 16 to 20, 16 to 19, 16 to 18, 16 to 17, 17 to 20, 17 to 19, 17 to 18, 18 to 20, 18 to 19, or 19 to 20 wt % of the oleogel external phase.

Emulsions can be characterized by volume fraction (Φ) of the internal (dispersed) phase.

In embodiments, the water-in-oil emulsion has an aqueous internal phase volume fraction (Φ) of from about 0.65 to 0.80, 0.65 to 0.79, 0.65 to 0.78, 0.65 to 0.77, 0.65 to 0.76, 0.65 to 0.75, 0.65 to 0.74, 0.65 to 0.73, 0.65 to 0.72, 0.65 to 0.71, 0.65 to 0.70, 0.65 to 0.69, 0.65 to 0.68, 0.65 to 0.67, 0.65 to 0.66, 0.66 to 0.80, 0.66 to 0.79, 0.66 to 0.78, 0.66 to 0.77, 0.66 to 0.76, 0.66 to 0.75, 0.66 to 0.74, 0.66 to 0.73, 0.66 to 0.72, 0.66 to 0.71, 0.66 to 0.70, 0.66 to 0.69, 0.66 to 0.68, 0.66 to 0.67, 0.67 to 0.80, 0.67 to 0.79, 0.67 to 0.78, 0.67 to 0.77, 0.67 to 0.76, 0.67 to 0.75, 0.67 to 0.74, 0.67 to 0.73, 0.67 to 0.72, 0.67 to 0.71, 0.67 to 0.70, 0.67 to 0.69, 0.67 to 0.68, 0.68 to 0.80, 0.68 to 0.79, 0.68 to 0.78, 0.68 to 0.77, 0.68 to 0.76, 0.68 to 0.75, 0.68 to 0.74, 0.68 to 0.73, 0.68 to 0.72, 0.68 to 0.71, 0.68 to 0.70, 0.68 to 0.69, 0.69 to 0.80, 0.69 to 0.79, 0.69 to 0.78, 0.69 to 0.77, 0.69 to 0.76, 0.69 to 0.75, 0.69 to 0.74, 0.69 to 0.73, 0.69 to 0.72, 0.69 to 0.71, 0.69 to 0.70, 0.70 to 0.80, 0.70 to 0.79, 0.70 to 0.78, 0.70 to 0.77, 0.70 to 0.76, 0.70 to 0.75, 0.70 to 0.74, 0.70 to 0.73, 0.70 to 0.72, 0.70 to 0.71, 0.71 to 0.80, 0.71 to 0.79, 0.71 to 0.78, 0.71 to 0.77, 0.71 to 0.76, 0.71 to 0.75, 0.71 to 0.74, 0.71 to 0.73, 0.71 to 0.72, 0.72 to 0.80, 0.72 to 0.79, 0.72 to 0.78, 0.72 to 0.77, 0.72 to 0.76, 0.72 to 0.75, 0.72 to 0.74, 0.72 to 0.73, 0.73 to 0.80, 0.73 to 0.79, 0.73 to 0.78, 0.73 to 0.77, 0.73 to 0.76, 0.73 to 0.75, 0.73 to 0.74, 0.74 to 0.80, 0.74 to 0.79, 0.74 to 0.78, 0.74 to 0.77, 0.74 to 0.76, 0.74 to 0.75, 0.75 to 0.80, 0.75 to 0.79, 0.75 to 0.78, 0.75 to 0.77, 0.75 to 0.76, 0.76 to 0.80, 0.76 to 0.79, 0.76 to 0.78, 0.76 to 0.77, 0.77 to 0.80, 0.77 to 0.79, 0.77 to 0.78, 0.78 to 0.80, 0.78 to 0.79, or 0.79 to 0.80.

As used herein, the term “Pickering crystals” refers to solid particles absorbed at the interface between two phases of an emulsion.

In an embodiment, the water-in-oil emulsion further comprises interfacial Pickering crystals on surfaces of aqueous phase droplets within the water-in-oil emulsion. Interfacial Pickering crystals are described in the art and may include, for example, crystals formed from plant materials (such as cellulose), or fat crystals formed from fats of various sources.

The water-in-oil emulsion can be characterized by the size of the droplets formed by the aqueous internal phase. In embodiments, these droplets have a mean particle diameter of about 10 to 30, 10 to 29, 10 to 28, 10 to 27, 10 to 26, 10 to 25, 10 to 24, 10 to 23, 10 to 22, 10 to 21, 10 to 20, 10 to 19, 10 to 18, 10 to 17, 10 to 16, 10 to 15, 10 to 14, 10 to 13, 10 to 12, 10 to 11, 11 to 30, 11 to 29, 11 to 28, 11 to 27, 11 to 26, 11 to 25, 11 to 24, 11 to 23, 11 to 22, 11 to 21, 11 to 20, 11 to 19, 11 to 18, 11 to 17, 11 to 16, 11 to 15, 11 to 14, 11 to 13, 11 to 12, 12 to 30, 12 to 29, 12 to 28, 12 to 27, 12 to 26, 12 to 25, 12 to 24, 12 to 23, 12 to 22, 12 to 21, 12 to 20, 12 to 19, 12 to 18, 12 to 17, 12 to 16, 12 to 15, 12 to 14, 12 to 13, 13 to 30, 13 to 29, 13 to 28, 13 to 27, 13 to 26, 13 to 25, 13 to 24, 13 to 23, 13 to 22, 13 to 21, 13 to 20, 13 to 19, 13 to 18, 13 to 17, 13 to 16, 13 to 15, 13 to 14, 14 to 30, 14 to 29, 14 to 28, 14 to 27, 14 to 26, 14 to 25, 14 to 24, 14 to 23, 14 to 22, 14 to 21, 14 to 20, 14 to 19, 14 to 18, 14 to 17, 14 to 16, 14 to 15, 15 to 30, 15 to 29, 15 to 28, 15 to 27, 15 to 26, 15 to 25, 15 to 24, 15 to 23, 15 to 22, 15 to 21, 15 to 20, 15 to 19, 15 to 18, 15 to 17, 15 to 16, 16 to 30, 16 to 29, 16 to 28, 16 to 27, 16 to 26, 16 to 25, 16 to 24, 16 to 23, 16 to 22, 16 to 21, 16 to 20, 16 to 19, 16 to 18, 16 to 17, 17 to 30, 17 to 29, 17 to 28, 17 to 27, 17 to 26, 17 to 25, 17 to 24, 17 to 23, 17 to 22, 17 to 21, 17 to 20, 17 to 19, 17 to 18, 18 to 30, 18 to 29, 18 to 28, 18 to 27, 18 to 26, 18 to 25, 18 to 24, 18 to 23, 18 to 22, 18 to 21, 18 to 20, 18 to 19, 19 to 30, 19 to 29, 19 to 28, 19 to 27, 19 to 26, 19 to 25, 19 to 24, 19 to 23, 19 to 22, 19 to 21, 19 to 20, 20 to 30, 20 to 29, 20 to 28, 20 to 27, 20 to 26, 20 to 25, 20 to 24, 20 to 23, 20 to 22, 20 to 21, 21 to 30, 21 to 29, 21 to 28, 21 to 27, 21 to 26, 21 to 25, 21 to 24, 21 to 23, 21 to 22, 22 to 30, 22 to 29, 22 to 28, 22 to 27, 22 to 26, 22 to 25, 22 to 24, 22 to 23, 23 to 30, 23 to 29, 23 to 28, 23 to 27, 23 to 26, 23 to 25, 23 to 24, 24 to 30, 24 to 29, 24 to 28, 24 to 27, 24 to 26, 24 to 25, 25 to 30, 25 to 29, 25 to 28, 25 to 27, 25 to 26, 26 to 30, 26 to 29, 26 to 28, 26 to 27, 27 to 30, 27 to 29, 27 to 28, 28 to 30, 28 to 29, or 29 to 30.

The water-in-oil emulsion can also be characterized by stability, measured as a function of one or more structural changes (e.g. in particle size, form, rheology, and/or phase separation) over a period of time, at a particular temperature). A suitable time would be within shelf-life range (in a scale of years). A suitable temperature would be a typical storage temperature of −20° C. to 80° C.

In an embodiment, stability is measured as the percent change of droplet size over time, where droplet size changes by less than about 20%, 19%, 18%, 17%, 16%, 15%, 14%, 13%, 12%, 11%, 10%, 9%, 8%, 7%, 6%, 5%, 4%, 3%, 2%, 1%, or 0.5%.

In an embodiment, stability is measured as change in form over time, where the original HIPE shape and/or height changes by less than about 20%, 19%, 18%, 17%, 16%, 15%, 14%, 13%, 12%, 11%, 10%, 9%, 8%, 7%, 6%, 5%, 4%, 3%, 2%, 1%, or 0.5%.

In an embodiment, rheological measurement is changed by more than about 5%, 6%, 7%, 8%, 9%, 10%, 11%, 12%, 13%, 14%, 15%, 16%, 17%, 18%, 19,%, or 20%.

Loss of stability can be measured by a decrease in internal (dispersed) phase volume fraction (Φ) over time, which is an indication of phase separation. Thus, retention of internal phase over time is an indicator of stability. In an embodiment, stability is measured as the proportion of internal phase retention over time, where the proportion of internal phase retained is at least 90%, 91%, 92%, 93%, 94%, 95%, 96%, 97%, 98%, or 99% of the original volume fraction (Φ) of the internal (dispersed) phase.

In embodiments, the emulsion is stable at from about −20 to 80, −20 to 70, −20 to 60, −20 to 50, −20 to 40, −20 to 30, −20 to 20, −20 to 10, −20 to 0, −20 to −10, −10 to 80, −10 to 70, −10 to 60, −10 to 50, −10 to 40, −10 to 30, −10 to 20, −10 to 10, −10 to 0, 0 to 80, 0 to 70, 0 to 60, 0 to 50, 0 to 40, 0 to 30, 0 to 20, 0 to 10, 10 to 80, 10 to 70, 10 to 60, 10 to 50, 10 to 40, 10 to 30, 10 to 20, 20 to 80, 20 to 70, 20 to 60, 20 to 50, 20 to 40, 20 to 30, 30 to 80, 30 to 70, 30 to 60, 30 to 50, 30 to 40, 40 to 80, 40 to 70, 40 to 60, 40 to 50, 50 to 80, 50 to 70, 50 to 60, 60 to 80, 60 to 70, or 70 to 80° C.

In embodiments, the emulsion is stable for at least 2, 3, 4, 5, 6, 7, 8, 9, 10, 11, 12, 13, 14, 15, 16, 17, 18, 19, 20, 21, 28, 35, 42, 49, 56, or 63 days.

In embodiments, the emulsion is stable for at least 0.5, 1, 1.5, 2, 2.5, 3, 3.5, 4, 4.5, 5, 5.5, or 6 years.

In an embodiment, the emulsion is stable at 25° C. for at least two months.

The phrase “hydrogel aqueous internal phase,” as used herein, refers to an aqueous based internal phase structured with an internal structurant to form a hydrogel.

In an embodiment, the internal structurant may be protein (non-limiting examples of which include collagen, elastin, fibrin, fibroin, gelatin, and globular proteins (e.g. bovine serum albumin, ß-lactoglobulin, ovalbumin)), a polysaccharide (non-limiting examples of which include agarose, alginate, carbomethylcellulose, carrageenan, chitosan, guar gum, gum acacia, hyaluronan, hyaluronic acid, starch, and xanthan gum), a biosynthetic polypeptide, an oligopeptide, a PEGylated polymer, or combinations thereof.

In an embodiment, the internal structurant is a carrageenan. In another embodiment, the carrageenan may be κ-carrageenan, ι-carrageenan, or λ-carrageenan. In embodiments of the present application, the internal structurant is present in an amount of from about 0.5 to 2.0 wt % of the hydrogel aqueous internal phase. In an embodiment, the internal structurant is present in an amount of from about 0.5 to 2.0, 0.5 to 1.9, 0.5 to 1.8, 0.5 to 1.7, 0.5 to 1.6, 0.5 to 1.5, 0.5 to 1.4, 0.5 to 1.3, 0.5 to 1.2, 0.5 to 1.1, 0.5 to 1.0, 0.5 to 0.9, 0.5 to 0.8, 0.5 to 0.7, 0.5 to 0.6, 0.6 to 2.0, 0.6 to 1.9, 0.6 to 1.8, 0.6 to 1.7, 0.6 to 1.6, 0.6 to 1.5, 0.6 to 1.4, 0.6 to 1.3, 0.6 to 1.2, 0.6 to 1.1, 0.6 to 1.0, 0.6 to 0.9, 0.6 to 0.8, 0.6 to 0.7, 0.7 to 2.0, 0.7 to 1.9, 0.7 to 1.8, 0.7 to 1.7, 0.7 to 1.6, 0.7 to 1.5, 0.7 to 1.4, 0.7 to 1.3, 0.7 to 1.2, 0.7 to 1.1, 0.7 to 1.0, 0.7 to 0.9, 0.7 to 0.8, 0.8 to 2.0, 0.8 to 1.9, 0.8 to 1.8, 0.8 to 1.7, 0.8 to 1.6, 0.8 to 1.5, 0.8 to 1.4, 0.8 to 1.3, 0.8 to 1.2, 0.8 to 1.1, 0.8 to 1.0, 0.8 to 0.9, 0.9 to 2.0, 0.9 to 1.9, 0.9 to 1.8, 0.9 to 1.7, 0.9 to 1.6, 0.9 to 1.5, 0.9 to 1.4, 0.9 to 1.3, 0.9 to 1.2, 0.9 to 1.1, 0.9 to 1.0, 1.0 to 2.0, 1.0 to 1.9, 1.0 to 1.8, 1.0 to 1.7, 1.0 to 1.6, 1.0 to 1.5, 1.0 to 1.4, 1.0 to 1.3, 1.0 to 1.2, 1.0 to 1.1, 1.1 to 2.0, 1.1 to 1.9, 1.1 to 1.8, 1.1 to 1.7, 1.1 to 1.6, 1.1 to 1.5, 1.1 to 1.4, 1.1 to 1.3, 1.1 to 1.2, 1.2 to 2.0, 1.2 to 1.9, 1.2 to 1.8, 1.2 to 1.7, 1.2 to 1.6, 1.2 to 1.5, 1.2 to 1.4, 1.2 to 1.3, 1.3 to 2.0, 1.3 to 1.9, 1.3 to 1.8, 1.3 to 1.7, 1.3 to 1.6, 1.3 to 1.5, 1.3 to 1.4, 1.4 to 2.0, 1.4 to 1.9, 1.4 to 1.8, 1.4 to 1.7, 1.4 to 1.6, 1.4 to 1.5, 1.5 to 2.0, 1.5 to 1.9, 1.5 to 1.8, 1.5 to 1.7, 1.5 to 1.6, 1.6 to 2.0, 1.6 to 1.9, 1.6 to 1.8, 1.6 to 1.7, 1.7 to 2.0, 1.7 to 1.9, 1.7 to 1.8, 1.8 to 2.0, 1.8 to 1.9, or 1.9 to 2.0 wt % of the hydrogel aqueous internal phase.

A third aspect of the present application is a composition comprising a water-in-oil emulsion comprising an aqueous internal phase, and an oleogel external phase comprising an oleaginous carrier, a surfactant, an external structurant, and one or more incorporated materials.

A fourth aspect of the present application is a composition comprising a water-in-oil emulsion comprising a hydrogel aqueous internal phase comprising an internal structurant, and an oleogel external phase comprising an oleaginous carrier, a surfactant, an external structurant, and one or more incorporated materials.

As used herein the phrase “incorporated material” refers to an ingredient or compound that is soluble or dispersible in any one of the aqueous internal phase, hydrogel aqueous internal phase, or oleogel external phase of a water-in-oil emulsion of the present application.

In an embodiment, the incorporated material is a hydrophilic compound incorporated or dispersed in the aqueous internal phase or the hydrogel aqueous internal phase. In another embodiment, the incorporated material is a hydrophobic compound incorporated or dispersed in the oleogel external phase. In an embodiment, the water-in-oil emulsions comprises two or more incorporated materials, with at least one of the incorporated materials being a hydrophilic compound incorporated or dispersed in the aqueous internal phase or the hydrogel aqueous internal phase and at least one of the incorporated materials being a hydrophobic compound incorporated or dispersed in the oleogel external phase.

In an embodiment, the incorporated material may be a pharmaceutical agent, a food agent, a cosmetic agent, or combinations thereof.

The term “pharmaceutical agent,” as used herein, encompasses all classes of chemical compounds exerting an effect in a biological system.

Non-limiting examples of pharmaceutical agents include DNA, RNA, oligonucleotides, polypeptides, peptides, antineoplastic agents, hormones, vitamins, enzymes, antivirals, antibiotics, anti-inflammatories, antiprotozoans, antirheumatics, radioactive compounds, antibodies, prodrugs, and combinations thereof.

The term “food agent,” as used herein, refers to an ingredient or compound that is fit for consumption by humans or other animals.

Food agents include, but are not limited to food products, food additives, dietary supplements, and combinations thereof.

In an embodiment, the food agent may be a protein, a protein-polysaccharide complex, a probiotic, a vitamin, an enzyme, an antioxidant, a colorant, a flavorant, an amino acid, a botanical, a fiber, an inulin, or combinations thereof.

In an embodiment, the food agent is selected from anthocyanin and ß-carotene. In another embodiment, the food agent is selected from whey protein and whey-protein polysaccharide complex.

Pharmaceutical agents or food agents may also encompass nutraceuticals.

The term “cosmetic agent,” as used herein, refers to an agent utilized, and/or intended to be applied to the human body for cleansing, beautifying, promoting attractiveness, altering the appearance of the skin or any combination thereof.

Cosmetic agents include, but are not limited to anti-acne agents, antidandruff agents, antimicrobial agents, antifungal agents, antioxidants, toners, skin conditioning or moisturizing agents, skin bleaching or lightening agents, hair conditioners, proteins, cleansers, oil control agents, skin care agents, anti-aging ingredients, sunscreen agents, sensation modifying agents, cooling agents, warming agents, relaxing or soothing agents, stimulating or refreshing agent, anti-itch ingredients, bug repellant ingredients, and combinations thereof.

In an embodiment, the composition of the present application has altered sensory properties compared to that of its incorporated material or materials alone. In another embodiment, the altered sensory property is astringency, and the astringency is reduced compared to that of the incorporated material alone.

In an embodiment, the incorporated material in the compositions of the present application has increased tolerance of acidity as compared that of the incorporated material alone.

In an embodiment, the incorporated material is a probiotic. In an embodiment, the probiotic retains viability for at least 1, 2, 3, 4, 5, 6, 7, 8, 9, 10, 11, 12, 13, 14, 30, 60, 90, 120, 360, or 720 days.

Another aspect of the present application is a method of delivering an incorporated material to a subject comprising selecting a subject in need of the incorporated material and administering, to the selected subject, a composition of the present application. Suitable compositions for the method are those described above.

Selecting a subject in need of the incorporated material may include, for example, diagnosis of a disease, illness, injury, or other physical condition in need of treatment. Diagnosis may include a formal physician's diagnosis or self-diagnosis. In this case, administering to the selected subject may include treating the diagnosed disease, illness, injury, or other physical condition in need of treatment. As used herein, the term “treating” refers to the application or administration of a composition of the present application to a subject, e.g., a patient. The treatment can be to cure, heal, alleviate, relieve, alter, remedy, ameliorate, palliate, improve or affect a disease, injury, illness, or other physical condition, the symptoms of a disease, injury, illness, or other physical condition, or the predisposition towards a disease, injury, or other physical condition.

Selecting a subject in need of the incorporated material may also include selecting a subject in need of nourishment. In this case, administering to the select subject may include feeding the selected subject.

The term “patient” and “subject” are interchangeable and may be taken to mean any living organism which may be administered and/or treated with compounds or compositions provided for herein. As such, the terms “patient” and “subject” may comprise, but is not limited to, any non-human mammal, primate or human. In some embodiments, the patient or subject is an adult, child or infant. In some embodiments, the patient or subject is a mammal. In some embodiments, the patient or subject is a human.

In methods of the present application, the administering step can be carried out systemically or via direct or local administration to a specific site. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of composition will vary depending on the type of incorporated material and the objective of the administration.

In an embodiment, the administering step is carried out topically. In another embodiment, the administering step is carried out in vivo.

As used herein, the term “topically” and “topical” refers to application of the compositions described herein to the surface of the skin, mucosal cells, tissues, and/or keratinous fibers.

Yet another aspect of the present application is a method of manufacturing a water-in-oil emulsion comprising a first step of mixing water with an oleaginous carrier under heat with constant high-shear homogenization to create a mixture, and a second step of cooling the mixture under continuous homogenization.

In an embodiment, the first step of mixing water with an oleaginous carrier is carried out at 70° C. In another embodiment, the first step of mixing water with an oleaginous carrier is carried out with a high-shear homogenization at 10,000 rpm. Other suitable heating temperatures will be the melting temperatures of the structurant(s), and a useful RPM will be in the range of 1-30,000 rpm.

In another embodiment, the second step of cooling the mixture is carried out at 25° C. In another embodiment, the second step of cooling the mixture is carried out with continuous homogenization at 10,000 rpm. Other suitable cooling temperatures can be any temperature below the structurant(s)'s melting temperature(s), and a useful RPM will be in the range of 1-30,000 rpm.

### EXAMPLES

The examples below are intended to exemplify the practice of embodiments of the disclosure but are by no means intended to limit the scope thereof.

### Examples 1-3: Ultra-Stable Water-in-Oil High Internal Phase Emulsions Featuring Interfacial and Biphasic Network Stabilization

### Materials and Methods for Examples 1-3

Materials. Fluorescein isothiocyanate isomers (FITC, >90.0%), Nile red, β-carotene (type I, synthetic, >93% purity), and sodium hydroxide (>98.0%) were purchased from Sigma-Aldrich (St. Louis, Mo., USA). Hydrochloric acid (36.5-38%) was obtained from VWR International (Radnor, Pa., USA). Canola oil was purchased from a local supermarket (Ithaca, N.Y., USA). Kappa (κ), iota (ι), and lambda (λ)-carrageenan was provided by TIC Gums Incorporated (White Marsh, Md., USA). GMO (Capmul GMO-50 EP/NF) was kindly donated by Abitec Corporation (Columbus, Ohio, USA). Beeswax was kindly donated by Strahl & Pitsch, Inc. (West Babylon, N.Y., USA). The anthocyanin used was obtained from blueberry extract from Bulk Supplements (Henderson, Nev., USA). All other chemicals used were of analytical grade.

Preparation of HIPEs.

In brief, the oil phase consisted of 1 wt % GMO in canola oil and was heated to 70° C. Distilled water at 70° C. was added slowly to the heated oil mixture with constant high-shear homogenization at 10,000 rpm (T25 digital Ultra Turrax, IKA Works, Wilmington, N.C., USA). With continuous homogenization, the HIPEs were submerged in an ice bath to cool down to 25° C. The internal phase volume fraction was calculated as the volume of the water phase included in the emulsion divided by the total volume of the emulsion. For O-HIPE, beeswax (0.5, 1, 3, 5, and 10 wt %) was additionally melted into the oil phase to increase the structure of the external phase. For gel-in-gel HIPEs, additional 1 wt % carrageenans (κ-/ι-/λ-) were incorporated in the water phase of the O-HIPE to increase the structure of the internal phase.

Rheological Measurements of HIPEs.

The O-HIPEs and gel-in-gel HIPEs were stored and measured at 25° C. Dynamic rheological measurements were conducted on an AR 1000 Rheometer (TA instruments, New Castle, Del., USA) using a 40-mm plate geometry and a gap of 500 The linear viscoelastic region was determined by a strain sweep at a frequency of 1 Hz from 0.0001 to 10. The G′ and G″ modulus was obtained through frequency sweeps from 0.1-10 rad/s using a fixed strain value of 0.0003.

Microscopy.

The microstructures of the HIPEs were inspected with a confocal laser scanning microscope (CLSM; LSM 710, Carl Zeiss, Gottingen, Germany) and a cryo-scanning electron microscope (cryo-SEM; FEI Strata 400S DualBeam focused ion beam/scanning electron microscope system FIB/SEM). For CLSM, the internal and external phases were stained with fluorescein isothiocyanate isomers (FITC) and Nile red at 1 mg/mL and 2 mg/mL, respectively. The HIPE structural morphology was studied using excitation/emission wavelengths of 488/515 nm and 492/518 nm for fluorescein isothiocyanate isomers and Nile red, respectively. For cryo-SEM imaging, the HIPEs were prepared by plunge freezing into slush nitrogen and transferred under vacuum into the system (Quorum PP3010T Cryo-FIB/SEM Preparation System, Quorum Technologies, Newhaven, UK). The sample was then maintained at −165° C., cross-sectioned with a fracturing knife, and coated with gold-palladium. Images were collected at 3 kV, with a working distance of 5 mm. Energy-dispersive X-ray spectroscopy (EDS) was performed during cryo-SEM imaging for spot element analysis. EDS was carried out using an accelerating voltage of 10 kV, and the data was collected and analyzed through INCA software (Oxford Instruments, Concord, Mass., USA).

Measurement of Contact Angle.

Contact angles of the internal phase on glass surfaces coated with 1% GMO and different concentrations of beeswax (0.5-10%) used in the external phase mixtures were measured using a tensiometer (ramé-hart model 500, Succasunna, N.J., USA). Approximately 5 μL of the internal phase was dispensed on the surface and contact angles were analyzed through DROPimage Advanced software (ramé-hart co., Succasunna, N.J., USA).

Particle Size Measurement.

To measure the mean particle diameters of the droplets within the HIPE samples, we utilized ImageJ software (v1.51, National Institute of Health, USA) for image analysis. This software calculates the droplet diameter through image pixel analysis. At least 100 particles were analyzed for each sample.

Physical Stability of HIPEs.

HIPEs were formed into a round disk-shape 1.5 cm in diameter and with a thickness of 0.5 cm. Images of these HIPE disks were taken after being dried for 2, 30, and 60 days under open-air at 25° C.

Anthocyanin and β-Carotene In Vitro Release.

To demonstrate the use of the resulting HIPEs as a potential nutraceutical/drug delivery system, anthocyanin-loaded HIPEs and anthocyanin-β-carotene co-loaded HIPEs were prepared to monitor the release of the anthocyanin and β-carotene under pH values of 1 and 5. Briefly, 1.25 mg/mL of the anthocyanin was incorporated into the internal phase at pH 5, and 1 mg/mL of the β-carotene was incorporated into the external phase. The anthocyanin-loaded HIPEs and anthocyanin-β-carotene co-loaded HIPEs were prepared using the same methods for HIPE fabrication described previously. The in vitro release of the anthocyanin and β-carotene were based on the membrane-free model with slight modifications (Tan et al., “Gelatin Particle-Stabilized High Internal Phase Emulsions as Nutraceutical Containers,” ACS Appl. Mater. Interfaces 6(16):13977-84 (2014), which is hereby incorporated by reference in its entirety). Approximately 100 mg of the HIPEs were weighed into glass vials and followed by the careful addition of water adjusted to pH 1 or 5. Simultaneously, canola oil was added into the vials, at which point the HIPEs reside between the water and oil interface. The glass vials were then shaken within a water bath at 37° C. At specific time intervals, aliquots of fluid from the water and oil phases were withdrawn and replaced by the same volumes of fresh water and oil medium. The amount of the released anthocyanin and β-carotene were measured using ultraviolet-visible (UV-Vis) spectrophotometry (UV-2600, Shimadzu Scientific Instrument, Marlborough, Mass., USA) at 520 nm and 452 nm, respectively (Tan et al., “Polyelectrolyte Complex Inclusive Biohybrid Microgels for Tailoring Delivery of Copigmented Anthocyanins,” Biomacromolecules 19(5):1517-27 (2018); and Tan et al., “Sonochemically-Synthesized Ultra-Stable High Internal Phase Emulsions via a Permanent Interfacial Layer,” ACS Sustain. Chem. Eng. 6(11):14374-82 (2018), which are hereby incorporated by reference in their entirety). The released amounts were calculated as the anthocyanin/β-carotene in the collected medium at a given time divided by the initial anthocyanin/β-carotene in the HIPEs and multiplied by 100. Each experiment was performed in triplicate, and the results were reported as mean±standard deviation.

### Example 1—Oleogel-HIPEs (O-HIPEs)

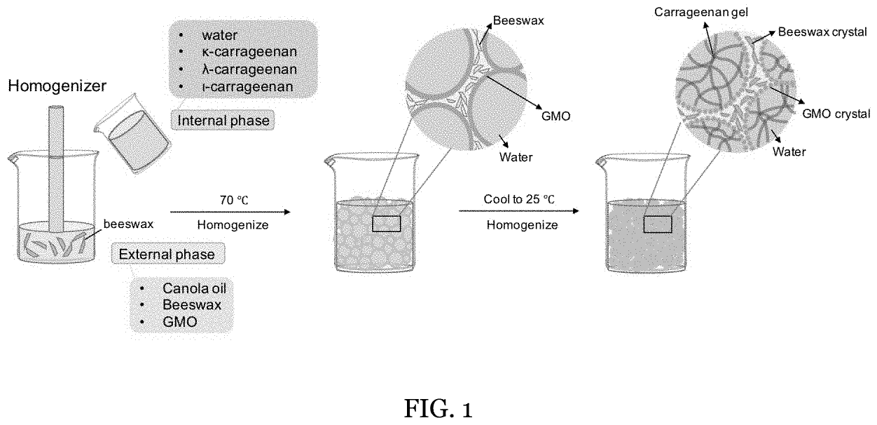

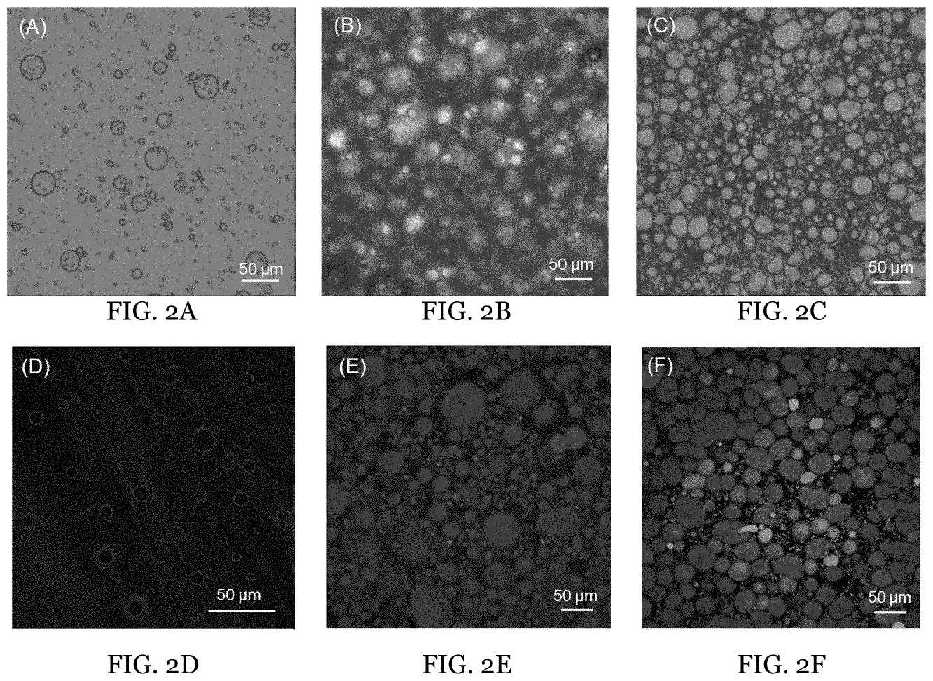

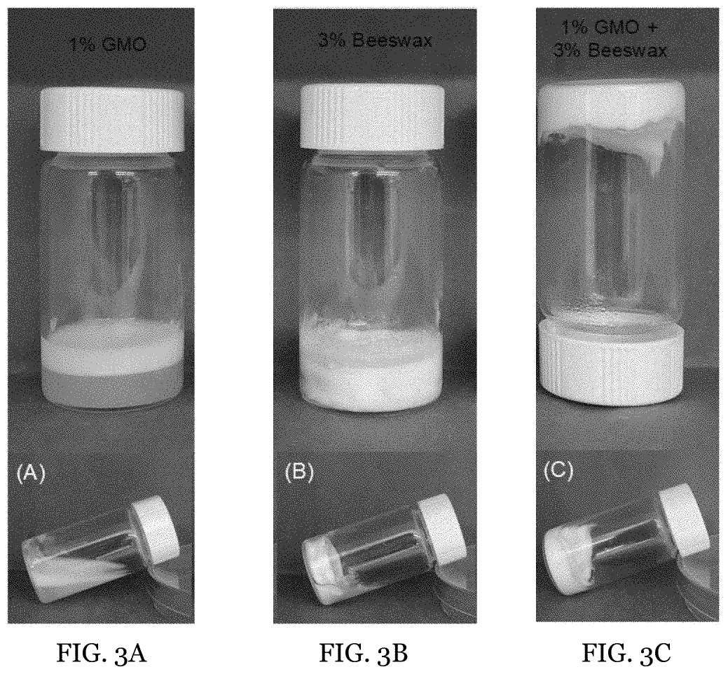

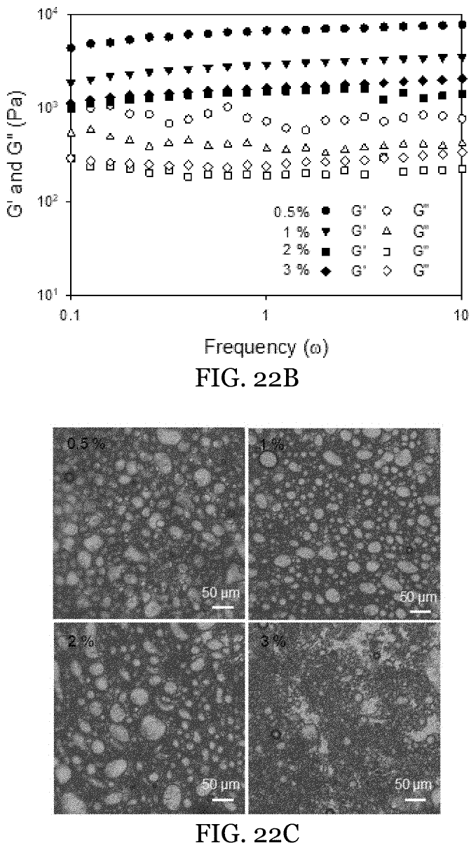

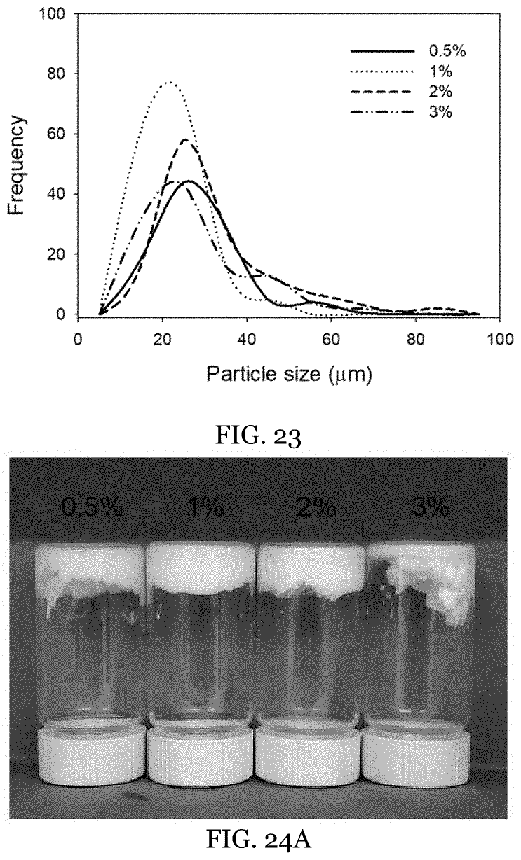

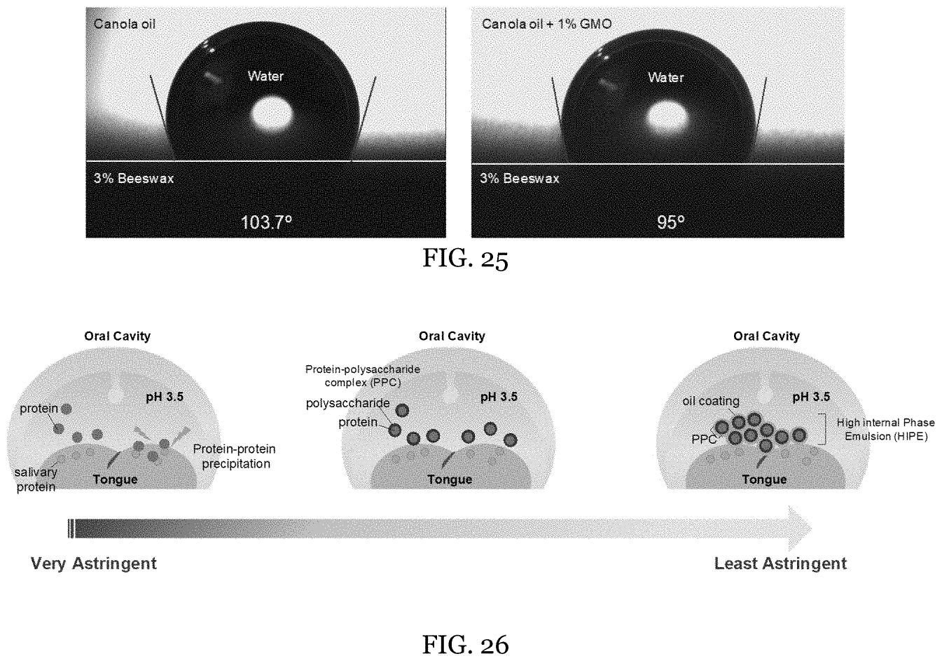

FIG. 1 shows the HIPE preparation process. Successful HIPE formation is dependent on the ability to incorporate more than 74 vol % of internal phase into the external phase. However, phase inversion occurs easily for W/O HIPEs due to the high interfacial surface tension and the low viscosity of the internal phase (Cameron et al., “High Internal Phase Emulsions (HIPEs)—Structure, Properties and Use in Polymer Preparation,” Advances in Polymer Science; Vol. 126, Berlin, Heidelberg: Springer pp 163-214 (1996), which is hereby incorporated by reference in its entirety). To overcome these limitations and form stable HIPEs, surfactants that are insoluble in the internal phase are typically required to lower the interfacial surface tension. Therefore, 1 wt % GMO, a natural biodegradable amphiphilic lipid was used as a surfactant to stabilize the W/O emulsions to form HIPEs by interfacial stabilization. However, lowering the interfacial surface tension alone does not promote W/O HIPE formation at Φ=0.75 (FIGS. 2A, 2D, 3A). Instead, phase inversion occurred, thus becoming an O/W emulsion, possibly due to low GMO surface activity, which is insufficient to stabilize high internal fractions. As demonstrated in FIGS. 22A-22C, more GMO (>=2 w %) in the external phase results in lower stability. This was also confirmed by particle size distribution. 1 w % GMO is optimal where it has a lower particle size distribution (FIG. 23). Previous research has reported similar outcomes when GMO is applied at higher concentrations of 5 wt % (Patel et al., “High Internal Phase Emulsion Gels (HIPE-Gels) Prepared Using Food-Grade Components,” RSC Adv. 4(35):18136-40 (2014), which is hereby incorporated by reference in its entirety). Alternatively, the use of a network stabilization method was explored, which involves increasing the structure and viscosity of the external phase to stabilize the internal phase. Therefore, 3 wt % beeswax was added to the external phase as a structurant to form an oleogel thin film between the internal phase droplets (FIGS. 2B, 2E). However, HIPEs made solely with beeswax have low stability, in which the dispersed phase showed a large average droplet diameter (28.86±11.33 μm) and phase separated over a day (FIG. 3B; Table 1). As a comparison, the HIPE formed by adding both surfactant (GMO) and structurant (beeswax) to the external phase increased the HIPE stability, as demonstrated by the smaller (17.59±6.18 μm) and more uniform size of the resulting droplets (FIGS. 2C, 2F, 3C; Table 1). The contact angle of water is lowered when both beeswax and GMO are in the external phase (FIG. 25). Therefore, a combination of GMO at low concentration and beeswax in the external phase endows the HIPE with both interfacial and network stabilization, a material referred to here as an oleogel-HIPE (O-HIPE), which features a single oleogel structured external phase.

TABLE 1
Particle size of HIPEs stabilized by solely 1 wt % GMO,
solely 3 wt % beeswax, and a combination of 1 wt % GMO
+ 3 wt % beeswax made at internal volume fractions
of 0.75. The values with different superscript letters
in a column are significantly different (p < 0.05).

| HIPE type | Emulsion type | Particle Size (μm) |
|---|---|---|
| GMO | O/W | N/A |
| Beeswax | W/O | 28.86 ± 11.33^{a} |
| GMO + Beeswax | W/O | 17.59 ± 6.18^{a} |

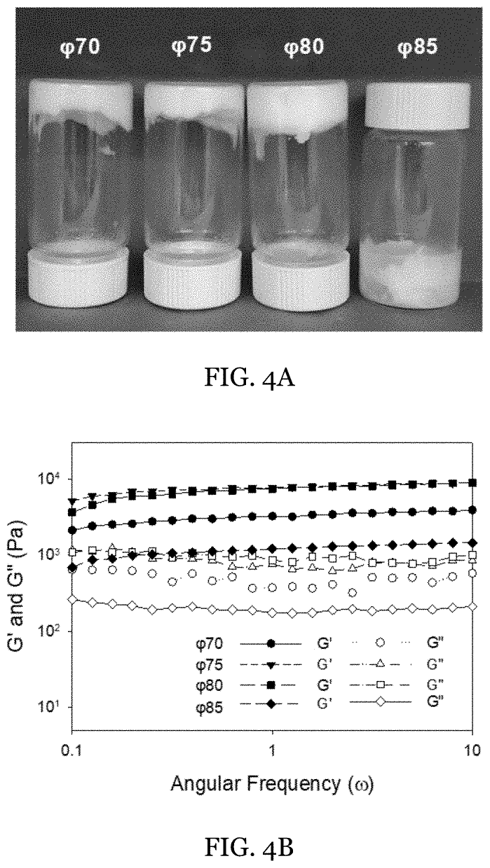

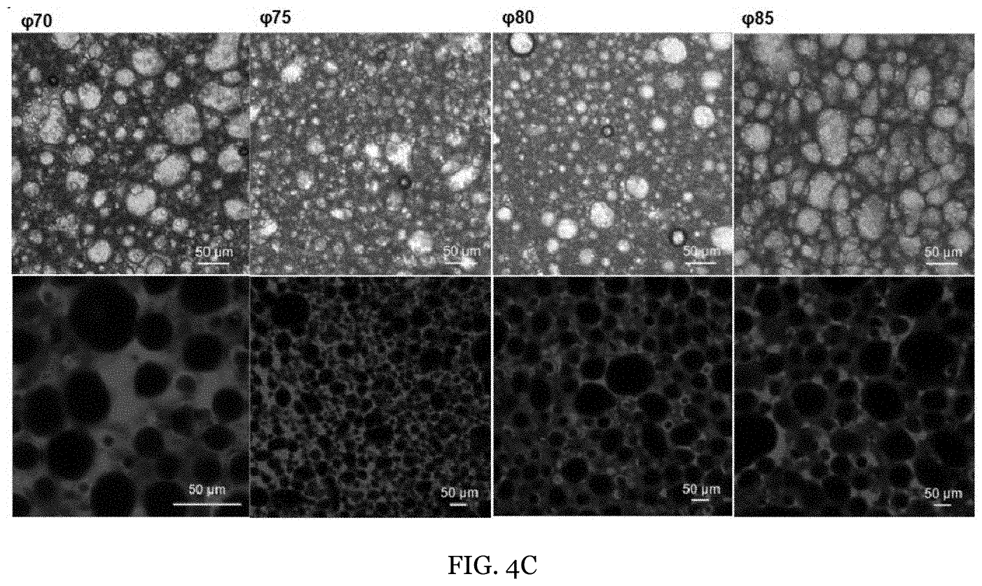

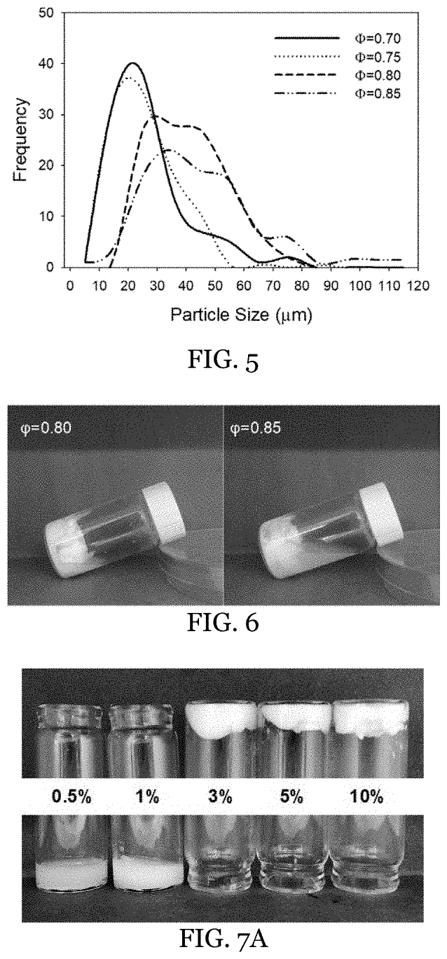

Next, the effect of different internal volume fractions (1) of water was investigated using fixed 1 wt % GMO and 3 wt % beeswax as stabilizers. As demonstrated in FIG. 4A, the highly concentrated emulsion can form gels with internal volume fractions of up to 0.80. Additionally, rheological measurements show that O-HIPEs with internal fractions of 0.75 and 0.80 demonstrate the highest storage modulus (G′) value, indicating stronger viscoelasticity behavior (FIG. 4B). However, at Φ=0.80 the droplet sizes are not the smallest (40.42±14.77) among all the samples prepared, but are the most tightly packed (FIG. 4C; Table 2). Interestingly, a viscoelastic O-HIPE was formed at Φ=0.70 even though the volume did not reach the packing density of Φ=0.74 (1). This was possibly due to the oleogel structure that increased the external phase viscosity, which lowered the packing density but still enabled a gel-like structure (Cameron et al., “High Internal Phase Emulsions (HIPEs)—Structure, Properties and Use in Polymer Preparation,” Advances in Polymer Science, Vol. 126, Berlin, Heidelberg: Springer, pp. 163-214 (1996); and Chen et al., “Effect of the Nature of the Hydrophobic Oil Phase and Surfactant in the Formation of Concentrated Emulsions,” J. Colloid Interface Sci. 145(1):260-9 (1991), which are hereby incorporated by reference in their entirety). Considering the microstructure of O-HIPEs with different internal fractions, the average particle sizes of the internal phase are smaller and more uniform at Φ=0.70 (27.16 μm) and Φ=0.75 (25.78 μm), with larger particle sizes and wider particle size distribution at Φ=0.80 (40.42 μm) and Φ=0.85 (45.77 μm) (FIG. 5; Table 2). Typically, a larger particle size would indicate the instability of the HIPEs overtime, as smaller particle sizes feature increased surface area and thus higher packing density and stability (Tan et al., “Sonochemically-Synthesized Ultra-Stable High Internal Phase Emulsions via a Permanent Interfacial Layer,” ACS Sustain. Chem. Eng. 6(11):14374-82 (2018), which is hereby incorporated by reference in its entirety). The instability that arises from larger droplet particle sizes was further shown in a storage study, in which it was observed that macroscopic separation occurred in O-HIPEs of Φ=0.80 and Φ=0.85 over 2 days of storage (FIG. 6). Therefore, a volume ratio of Φ=0.75 was selected for all subsequent emulsions studied.

TABLE 2
Particle size of O-HIPEs made from different internal phase
volume ratios. The value with different superscript letters
in a column are significantly different (p < 0.05).

| | Internal phase volume ratio (Φ) | | | |
|---|---|---|---|---|
| | 0.70 | 0.75 | 0.80 | 0.85 |
| Particle Size | 27.16 ± 13.41^{a} | 25.78 ± 11.22^{a} | 40.42 ± 14.77^{b} | 45.77 ± 21.01^{b} |
| (μm) | | | | |

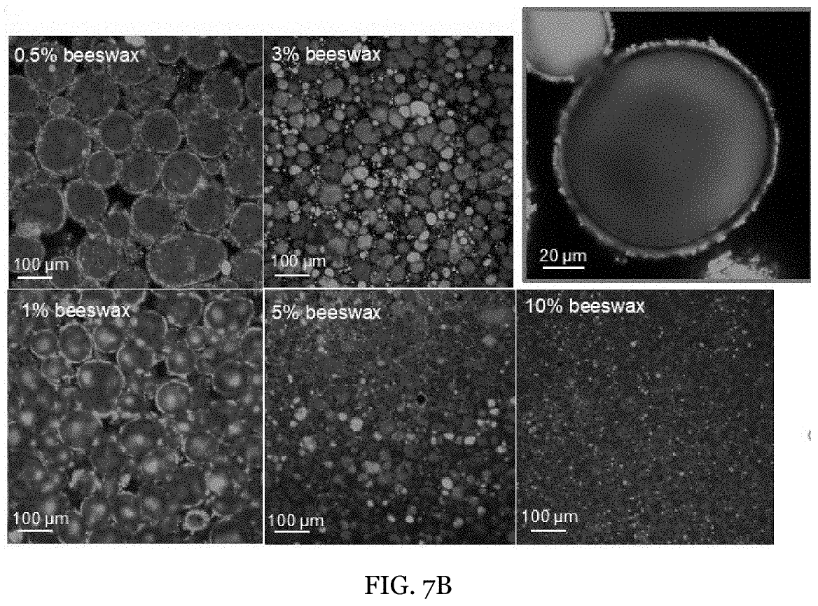

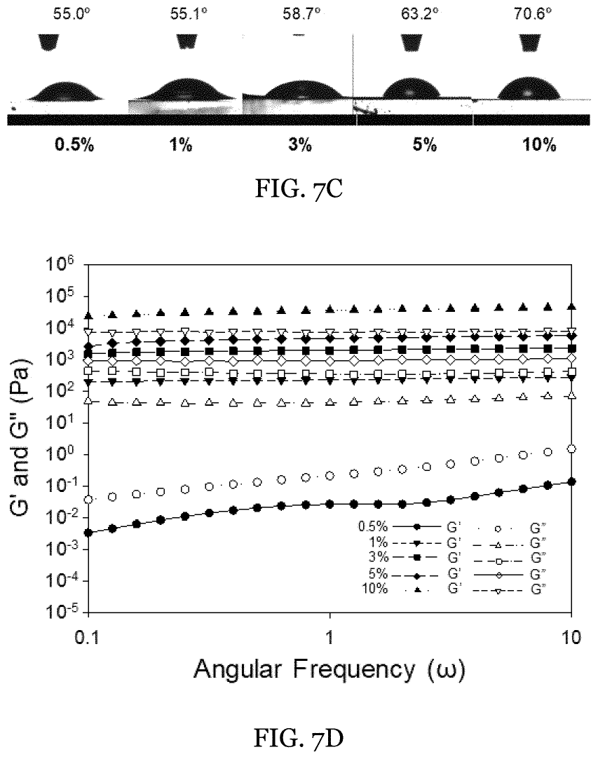

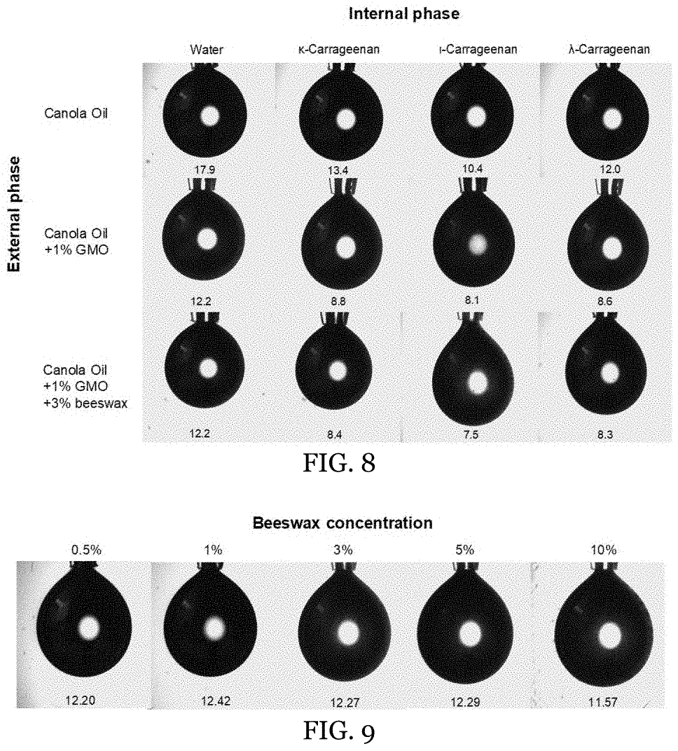

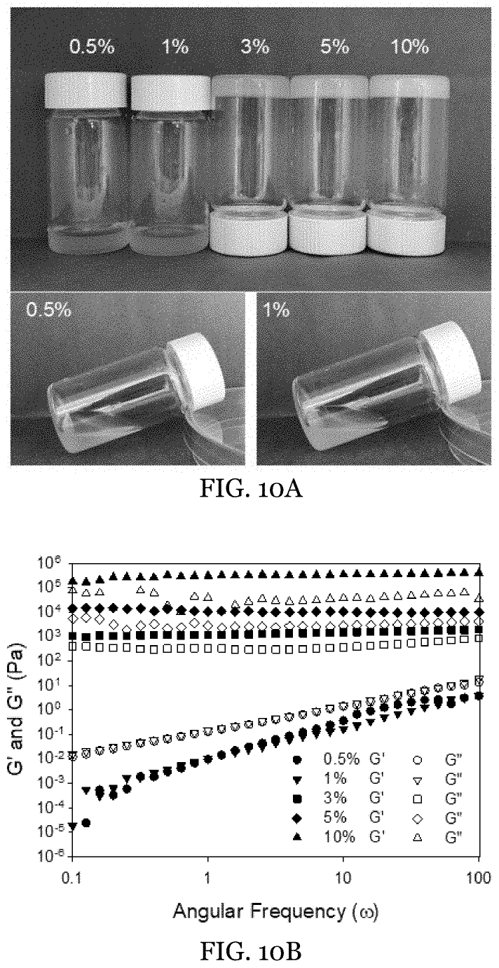

Mechanically, a more viscous and rigid external phase can increase the O-HIPE stability but will result in a lower maximum internal phase volume (Cameron et al., “High Internal Phase Emulsions (HIPEs)—Structure, Properties and Use in Polymer Preparation,” Advances in Polymer Science, Vol. 126, Berlin, Heidelberg: Springer, pp. 163-214 (1996); Chen et al., “Effect of the Nature of the Hydrophobic Oil Phase and Surfactant in the Formation of Concentrated Emulsions,” J. Colloid Interface Sci. 145(1):260-9 (1991); and Lee et al., “Combination of Internal Structuring and External Coating in an Oleogel-Based Delivery System for Fish Oil Stabilization,” Food Chem. 277 (2019), which are hereby incorporated by reference in their entirety). Therefore, the effect of beeswax concentration on the rheological behavior and overall ability to form O-HIPEs was studied. FIG. 7A shows that at Φ=0.75, a beeswax concentration of greater than 3 wt % is necessary for the formation of the O-HIPE. The microstructure of the O-HIPE also varied with beeswax concentration (FIG. 7B). Strikingly, the high magnification image of a single aqueous droplet under CLSM showed a distinct interfacial layer formed with surface-active particles, suggesting that these O-HIPEs are potentially Pickering HIPEs (FIG. 7B inset). This observation is similar to a previous study, demonstrating that at high temperature GMO dissolves in the oil external phase and then forms GMO crystals around the aqueous droplets when cooled (Macierzanka et al., “Effect of Crystalline Emulsifier Composition on Structural Transformations of Water-in-Oil Emulsions: Emulsification and Quiescent Conditions,” Colloids Surfaces A Physicochem. Eng. Asp. 334(1-3):40-52 (2009), which is hereby incorporated by reference in its entirety). Interestingly, these GMO crystals can originate from the external phase as pre-formed crystals and/or act as surfactant, which solidifies at the droplet interface (Ghosh et al., “Fat Crystals and Water-in-Oil Emulsion Stability,” Curr. Opin. Colloid Interface Sci. 16(5):421-31 (2011); and Rousseau, “Fat Crystals and Emulsion Stability—A Review,” Food Res. Int. 33(1):3-14 (2000), which are hereby incorporated by reference in their entirety). Additionally, previous research had demonstrated molten wax's role in W/O emulsions, in which rapidly cooling waxes contribute to the increased structure of the external phase, thus endowing network stabilization (Binks et al., “Effects of Temperature on Water-in-Oil Emulsions Stabilised Solely by Wax Microparticles,” J. Colloid Interface Sci. 335(1):94-104 (2009), which is hereby incorporated by reference in its entirety). This was evident in interfacial tension measurements, where GMO addition lowers the internal phase surface tension but beeswax addition does not (FIGS. 8 and 9). Therefore, with a fixed GMO concentration of 1 wt %, FIGS. 7A-7D demonstrates beeswax's main role in external network building. The effect of beeswax concentration on the network stabilization can be demonstrated by measurement of contact angle (FIG. 7C) and bulk oleogel properties (FIG. 10). The contact angle was measured by dispensing water on different oleogel films made with 1 wt % GMO and beeswax concentrations of 0.5-10 wt % (FIG. 7C). The lowest contact angle was observed at 0.5-1 wt % beeswax (55.0°), which slightly increased at 3 wt % (58.7°), followed by a steady increase at 5 wt %(63.2°) and 10 wt % (70.6°). Water wetted the oleogel film to a more noticeable degree at 0.5-1% beeswax, demonstrating the flowability of both phases and the low external network. For bulk oleogel, beeswax was incorporated in the oil to increase the network (FIG. 10). It was determined that a cutoff point of 3 wt % beeswax was necessary to form a self-standing bulk oleogel (FIG. 10A) and observed increased storage moduli (G′) at higher beeswax concentration (FIG. 10B), which is consistent with the rheological trend of the O-HIPEs (FIG. 7D). Together, it is evident that the O-HIPEs are dependent on the strength of the external network. With low external network, such as 0.5-1 wt % beeswax, O-HIPEs cannot be formed. However, for high external networks, such as 10 wt % beeswax, the O-HIPEs are not sufficiently stable.

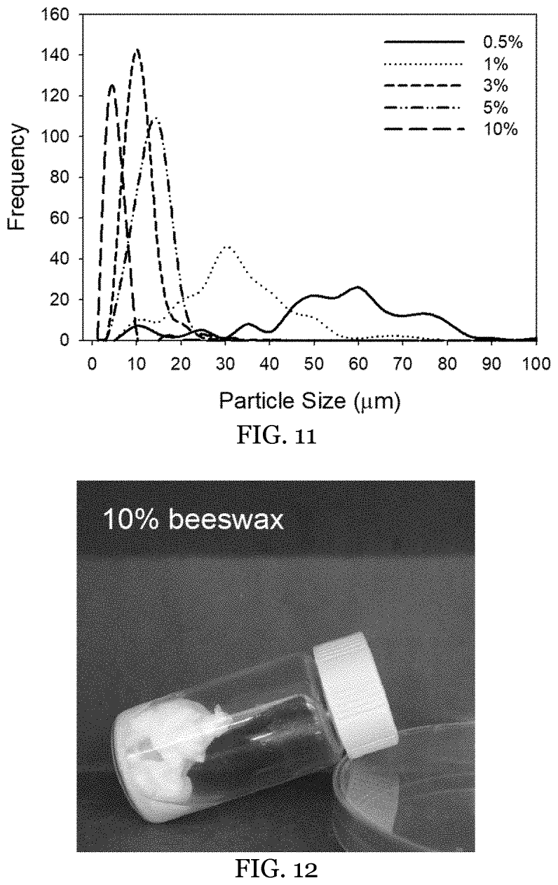

It was also found that the beeswax concentration affected the particle size and size distribution (FIG. 11; Table 3). 0.5 and 1 wt % beeswax in the external phase showed multimodal particle distributions and large particle sizes, suggesting the inability to form O-HIPEs. When studying the rheological behavior of the O-HIPEs, it was observed that G′ increases with increasing beeswax concentration (FIG. 7D). Due to the lack of external structure, 0.5 wt % beeswax resulted in the lowest G′, and in which G″>G′, indicating more liquid-like behavior (Lee et al., “Combination of Internal Structuring and External Coating in an Oleogel-Based Delivery System for Fish Oil Stabilization,” Food Chem. 277 (2019), which is hereby incorporated by reference in its entirety). Although the O-HIPE with 10 wt % beeswax showed the highest G′, suggesting the best stability, it was not stable as phase separation occurred when stored over 2 days (FIG. 12). This is due to the extensive external network provided by the 10 wt % beeswax upon cooling, the high viscosity of which prevents efficient homogenization during fabrication, thus lowering the amount of internal phase that can be incorporated (Cameron et al., “High Internal Phase Emulsions (HIPEs)—Structure, Properties and Use in Polymer Preparation,” Advances in Polymer Science, Vol. 126, Berlin, Heidelberg: Springer, pp. 163-214 (1996); and Chen et al., “Effect of the Nature of the Hydrophobic Oil Phase and Surfactant in the Formation of Concentrated Emulsions,” J. Colloid Interface Sci. 145(1):260-9 (1991), which are hereby incorporated by reference in their entirety). Therefore, the inventors believe that the highest viscoelastic behavior for O-HIPEs with 10 wt % beeswax is a result of the excess bulk beeswax network in the external phase. In addition, O-HIPEs formed with 5 wt % beeswax did not show a higher rheological performance as compared with samples made with 3 wt % beeswax. This cutoff threshold indicates an optimal beeswax concentration of 3 wt % (FIG. 7D). Overall, formation of O-HIPEs by combining Pickering GMO and a beeswax network for high internal aqueous phase stabilization was demonstrated.

TABLE 3
Particle size of O-HIPEs made from different beeswax concentrations. The values with
different superscript letters in a column are significantly different (p < 0.05).

| | Beeswax concentration (wt %) | | | | |
|---|---|---|---|---|---|
| | 0.5 | 1 | 3 | 5 | 10 |
| Particle Size | 105.25 ± 38.34^{a} | 59.74 ± 23.68^{b} | 16.50 ± 6.42^{c} | 21.90 ± 7.72^{d} | 5.75 ± 1.62^{e} |
| (μm) | | | | | |

### Example 2—Gel-in-Gel HIPEs

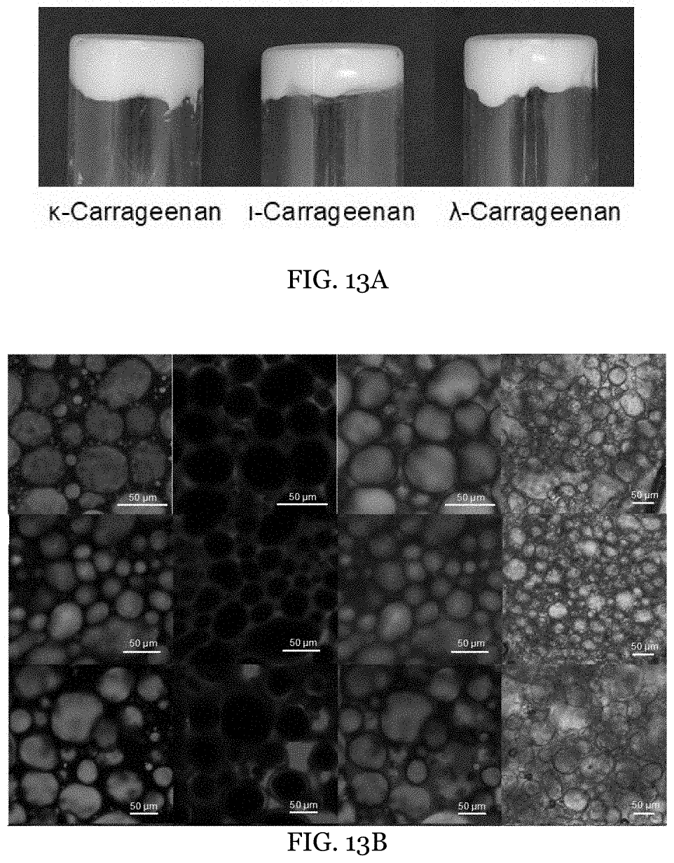

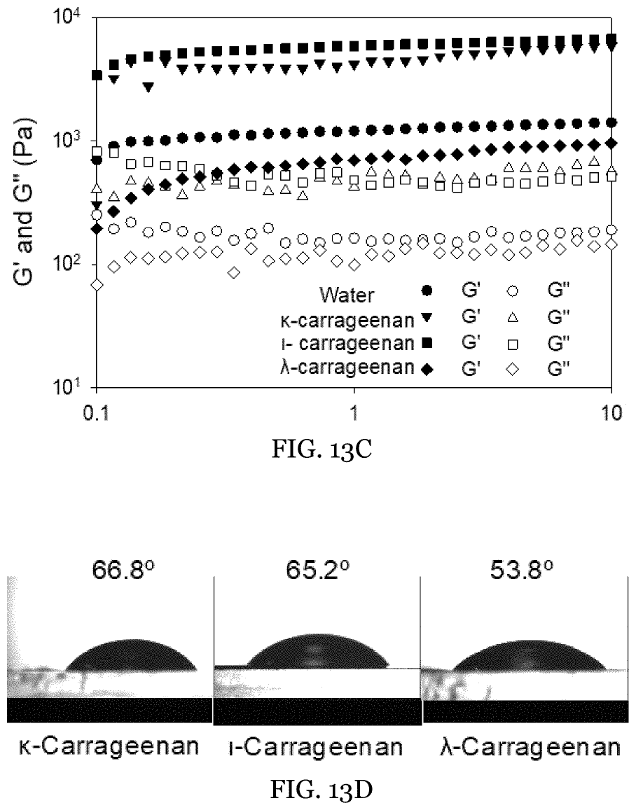

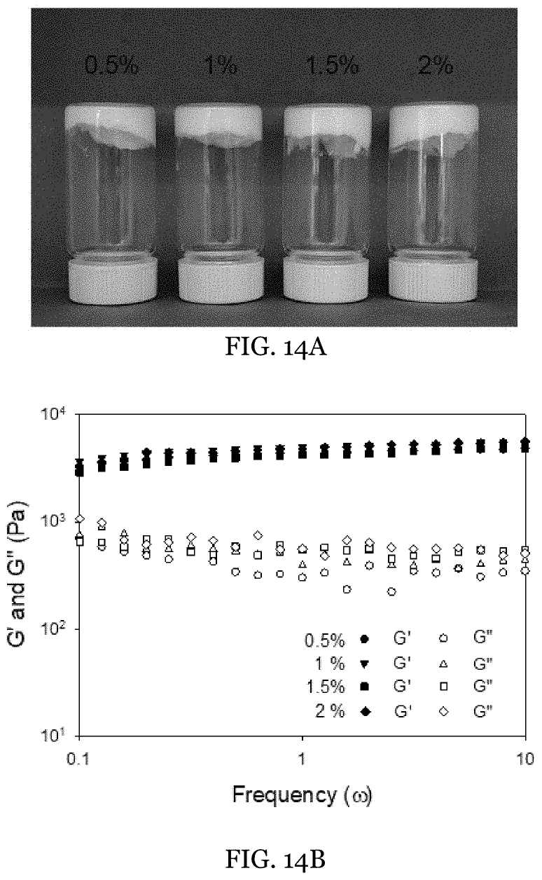

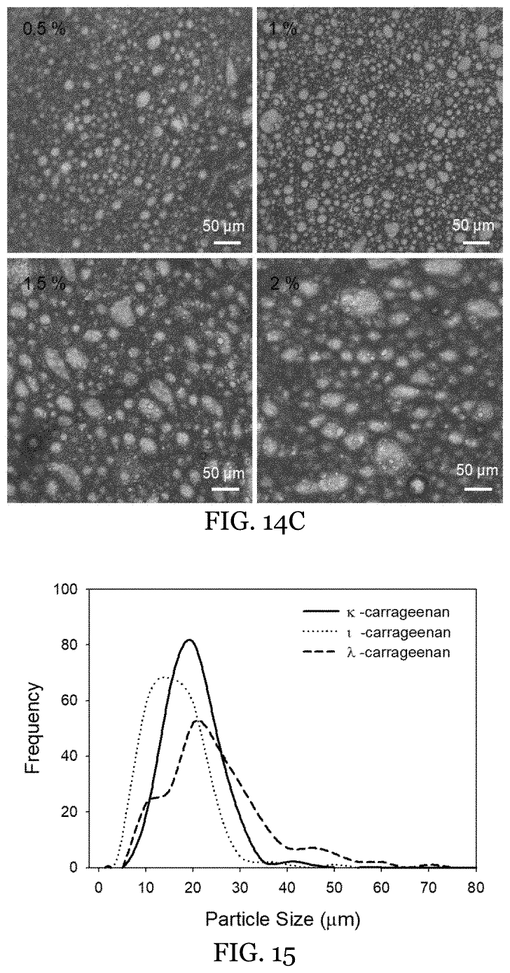

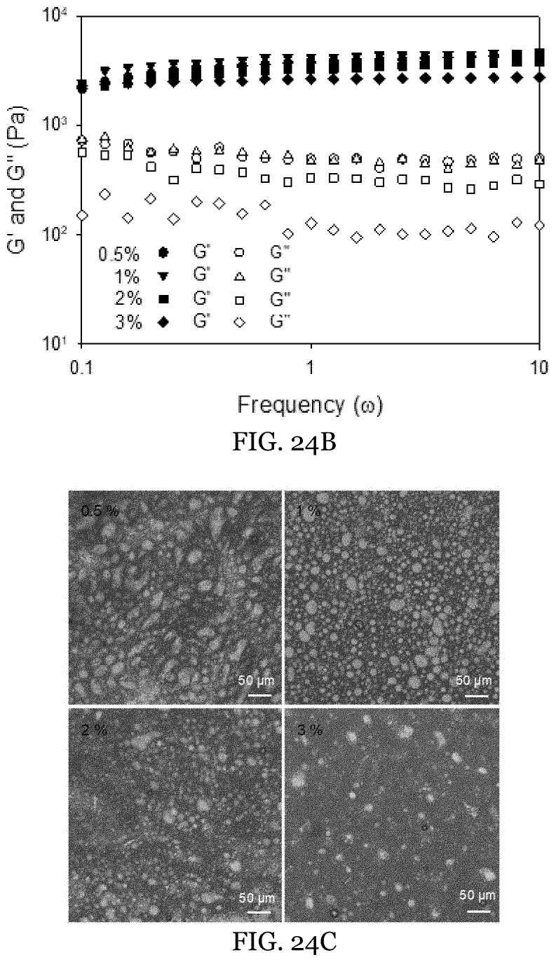

In addition to structuring the external phase, the network of the internal phase was further increased using carrageenans, which are polysaccharides typically used as thickening and gelling agents (Campo et al., “Carrageenans: Biological Properties, Chemical Modifications and Structural Analysis—A Review,” Carbohydrate Polymers 77(2):167-80 (2009), which is hereby incorporated by reference in its entirety). Such HIPEs, with both phases structured, are referred to here as gel-in-gel HIPEs. Building upon the most stable O-HIPE containing 3 wt % beeswax, the internally-structured HIPEs can be successfully formed with various types of carrageenan, including κ-, ι-, and λ-carrageenan (FIG. 13A) at 1 wt % (FIGS. 14A-14C). When increasing carrageenan concentration, there was slight increase in rheological behavior. Higher carrageenan concentration in the internal phase resulted in increased internal droplet packing (FIGS. 24A-24C). Microscopy imaging reveals that the gel-in-gel HIPEs formed with ι-carrageenan feature the smallest droplet sizes (27.18±12.95 μm) and the highest packing morphologies, while those formed with κ-carrageenan and λ-carrageenan showed similar packing microstructures, but with larger particle sizes (FIG. 13B; Table 4).

TABLE 4
Particle size of gel-in-gel HIPEs made from different carrageenans
in the internal phase. The values with different superscript
letters in a column are significantly different (p < 0.05).

| | Internal Phase (Carrageenan) | | |
|---|---|---|---|
| | κ- | ι- | λ- |
| Particle Size | 34.90 ± 11.40^{a} | 27.18 ± 12.95^{b} | 45.61 ± 25.72^{c} |
| (μm) | | | |

In terms of the rheological performance (FIG. 13C), ι-carrageenan endowed the gel-in-gel HIPE with the strongest viscoelasticity, followed by κ-carrageenan. By increasing the network in the internal phase, gel-in-gel HIPEs can increase the viscoelastic behavior by more than 5-fold compared to the corresponding O-HIPE, except for λ-carrageenan, which features a G′ value even lower than the O-HIPE. This may be explained by the fact that λ-carrageenan is the most sulfated carrageenan and has a flat structure, which makes it a non-gelling thickening agent (Campo et al., “Carrageenans: Biological Properties, Chemical Modifications and Structural Analysis—A Review,” Carbohydrate Polymers 77(2):167-80 (2009), which is hereby incorporated by reference in its entirety). In addition, the shear-thinning characteristics of λ-carrageenan enable it to be incorporated into the gel-in-gel HIPE's internal phase, but it is incapable of maintaining the rigid structure over time and wider ranges of the particle size distribution are observed (FIG. 15). Interestingly, the contact angles of pure κ- and ι-carrageenan are higher while λ-carrageenan is lower than water on a 3 wt % beeswax oleogel film (FIGS. 7C, 13D). The lower contact angle measurement suggests that λ-carrageenan has more affinity toward the external phase, leading to the instability of the dispersed aqueous phase.

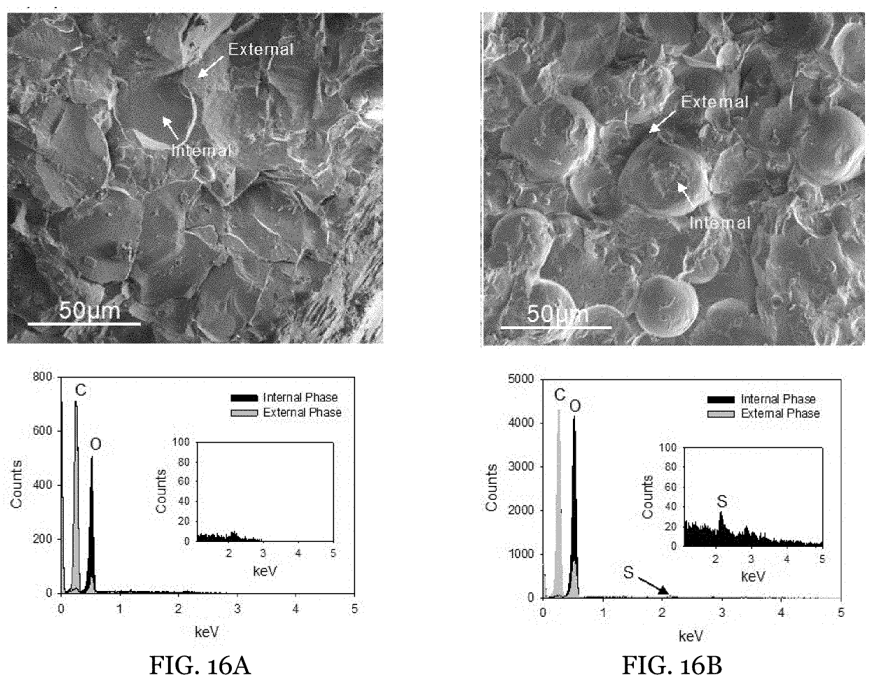

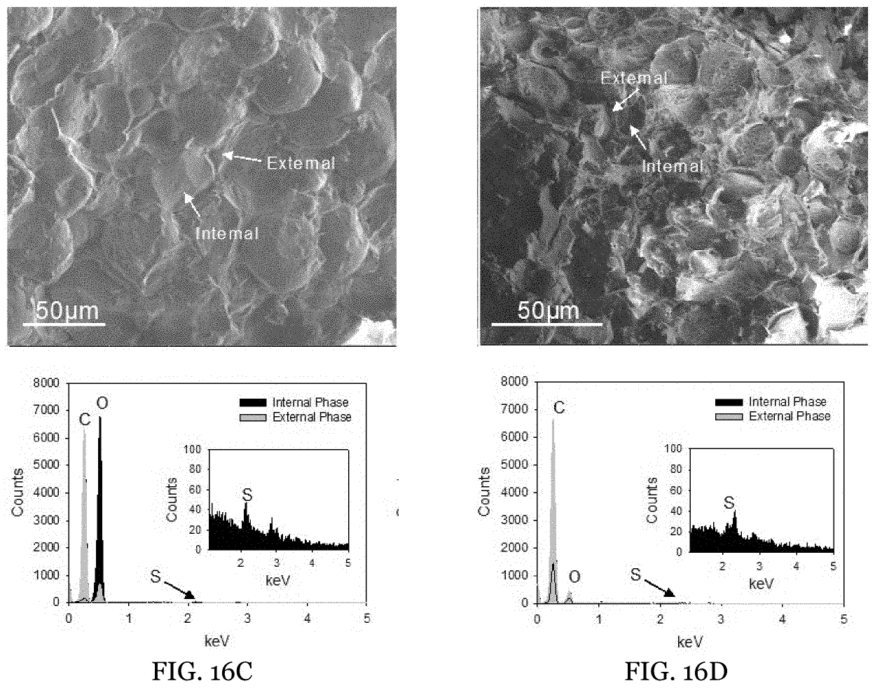

The formation of these gel-in-gel HIPEs is attributed to the increased structuring in both phases. To better understand the HIPE structure and the materials at each phase, cryo-SEM imaging and EDS elemental analysis were performed, respectively. FIGS. 16A-16D show the packing of the O-HIPE and gel-in-gel HIPE droplets with defined borders between the internal and external phases and a clear visualization of the droplet structure. The gel-in-gel HIPEs formed with λ-carrageenans showed less distinct droplet morphologies, which would result in less packing and thus lower stability, which is consistent with the sample's rheological behavior (FIG. 13C). EDS analysis was also performed on the apparent internal and external phase regions. Theoretically, in W/O HIPEs the internal phase should be composed of a higher oxygen content due to the greater presence of water compared to the external phase, which should be composed mostly of carbon from canola oil and beeswax. Table 5 displays approximate atomic percentages of each detected element from the EDS analysis. It was found that the internal phase was mostly composed of oxygen, whereas the external phase consisted of mostly carbon. Although both the internal and external phases of the λ-carrageenan sample showed higher carbon content compared to oxygen, a significant sulfur peak appeared in the analysis of the internal phase (FIG. 16D inset). This sulfur peak is from λ-carrageenan, which contains 3 sulfur groups per 2 sugar molecules in its chemical structure. Here, it was confirmed that carrageenans are trapped within the internal phase, which greatly assist in internal network stabilization.

TABLE 5
EDS elemental analysis of carbon, oxygen,
and sulfur from the cryo-SEM.

| | Internal Phase (Atomic %) | | | | External Phase (Atomic %) | | | |
|---|---|---|---|---|---|---|---|---|
| Element | Water | κ-car | ι-car | λ-car | Water | κ-car | ι-car | λ-car |
| C | 2.77 | 2.27 | 5.62 | 80.37 | 89.19 | 79.99 | 83.28 | 89.32 |
| O | 97.23 | 97.30 | 94.21 | 18.12 | 10.81 | 19.70 | 15.94 | 10.68 |
| S | — | — | — | 0.58 | — | — | — | — |

### Example 3—Stability Assessment

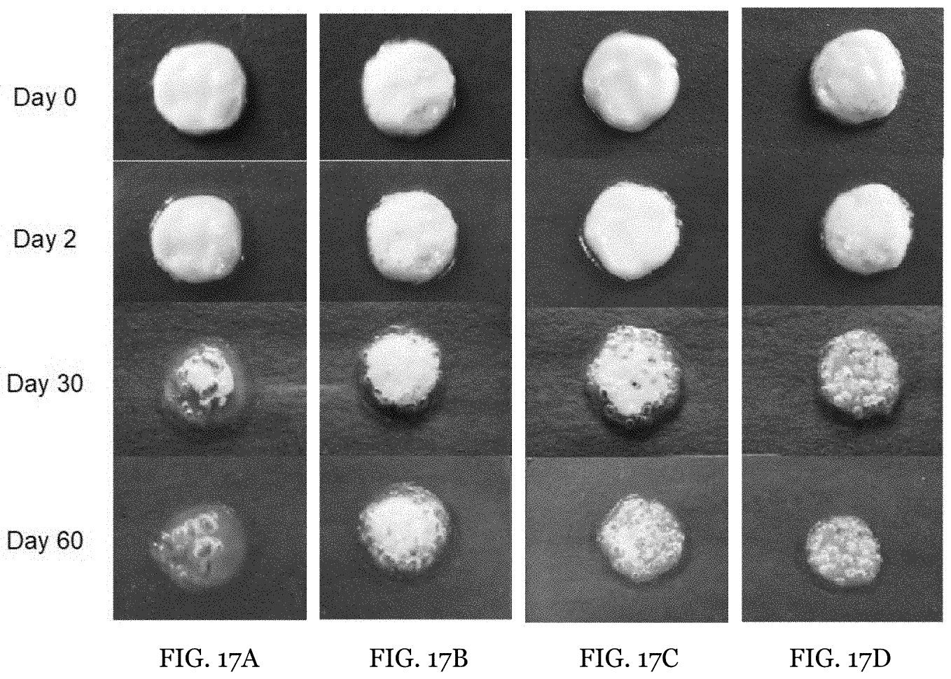

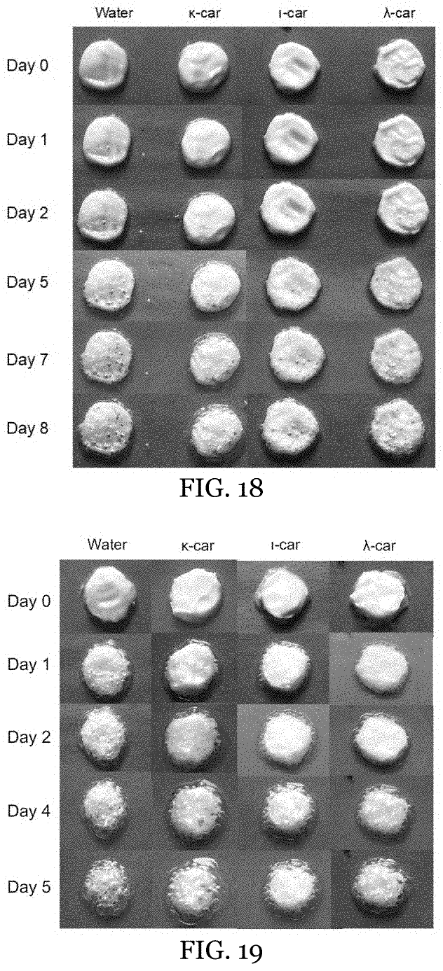

Physical stability. It is essential to investigate the stability of emulsions during a given storage period, as emulsions are thermodynamically unstable with a tendency to coalesce. FIGS. 17A-17D demonstrate the stability of the O-HIPE and gel-in-gel HIPEs over 2 months at 25° C. in open atmosphere. It was observed that the O-HIPE remained stable up to day 2 but collapsed after 30 days with a transparent appearance. In contrast, the gel-in-gel HIPEs maintained their structure, remaining opaque over 2 months, indicating the presence of an emulsion structure (Chantrapornchai et al., “Influence of Flocculation on Optical Properties of Emulsions, J. Food Sci. 66(3):464-9 (2001), which is hereby incorporated by reference in its entirety). The inventors attribute the differences in stability to the interfacial and network stabilization of the gel-in-gel HIPEs, in which the internal aqueous droplets with high viscosity are less prone to coalescence and are protected by an external layer of solid oil, which prevents evaporation. Results from accelerated storage studies under vacuum (FIG. 18) and 37° C. (FIG. 19) are concordant. This excellent stability of the gel-in-gel HIPEs is due to the tightly packed internal phase that is internally stabilized by carrageenans, interfacially stabilized by the GMO, and externally stabilized by the beeswax network.

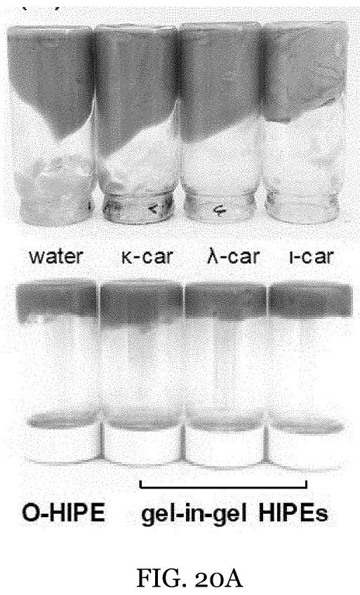

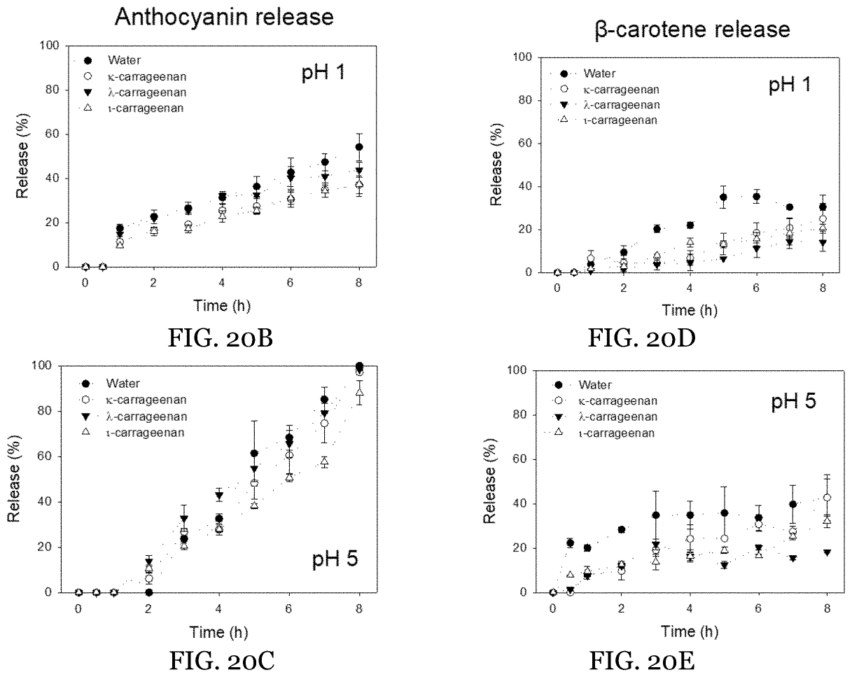

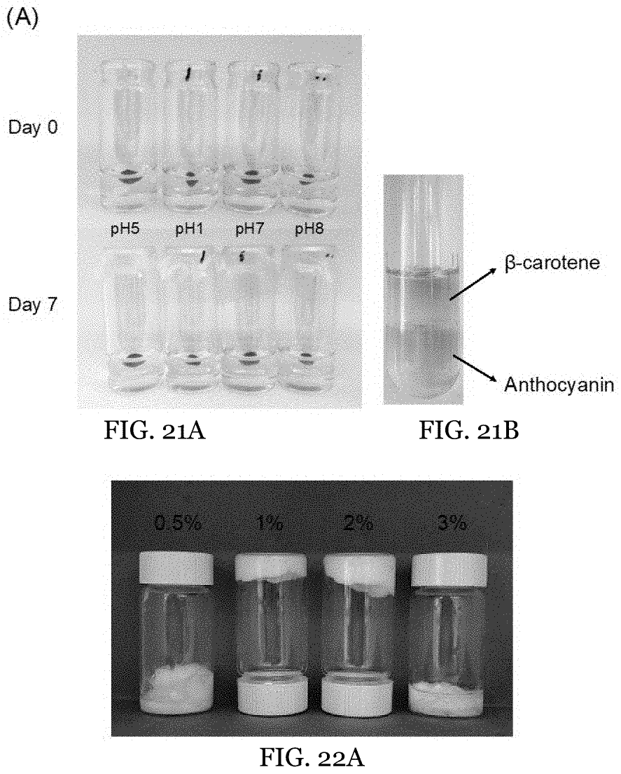

In vitro release of anthocyanin and β-carotene. The present strategy for forming ultra-stable HIPEs involves a structured biphasic system of both oil and aqueous phases, and unlike conventional W/O emulsions consisting of a liquid-in-liquid model with more flowability, this unique gel-in-gel HIPE enables loading of both hydrophilic and hydrophobic nutraceuticals immobilized in the internal and external phases, respectively. To understand the responsiveness of the gel-in-gel HIPEs under pH shock, anthocyanin was used as a model hydrophilic bioactive due to its health benefits, but also its high sensitivity to pH. At acidic pH, anthocyanins are red, and gradually shift to a purple to blue color as pH value increases (Wrolstad, “Anthocyanin Pigments-Bioactivity and Coloring Properties,” J. Food Sci. 69(5):C419-C425 (2006); and Sui et al., “Combined Effect of PH and High Temperature on the Stability and Antioxidant Capacity of Two Anthocyanins in Aqueous Solution,” Food Chem. 163:163-70 (2014), which are hereby incorporated by reference in their entirety). It was hypothesized that with the help of biphasic structuring in the present gel-in-gel HIPE, the entrapped anthocyanin would be greatly protected against environmental changes. FIG. 20A shows the anthocyanin-incorporated O-HIPE and gel-in-gel HIPEs. At 25° C., the anthocyanin-containing HIPEs showed no color change over 7 days of storage in solutions of different pHs (1-8) (FIG. 21A). This outstanding color stability is comparable to other color stabilization methods, such as layer-by-layer encapsulation (Tan et al., “Polyelectrolyte Microcapsules Built on CaCO3 Scaffolds for the Integration, Encapsulation, and Controlled Release of Copigmented Anthocyanins,” Food Chem. 246:305-12 (2018), which is hereby incorporated by reference in its entirety) and copigmentations (Tan et al, “Anthocyanin Stabilization by Chitosan-Chondroitin Sulfate Polyelectrolyte Complexation Integrating Catechin Co-Pigmentation,” Carbohydr. Polym. 181:124-31 (2018), which is hereby incorporated by reference in its entirety), as anthocyanin is highly unstable at pH 8. At a higher temperature (37° C.) simulating the human body, anthocyanin releases slowly, as such temperature approaches the melting temperature of internal carrageenans (40° C.). Despite partial disintegration of carrageenans, up to 45-70% of the original anthocyanin remained after 8 h at pH 1 (FIG. 20B). It was also observed that gel-in-gel HIPEs made with κ-carrageenan and ι-carrageenan retained anthocyanin the best, while λ-carrageenan retained anthocyanin similarly to the O-HIPE. At pH 5, the release of anthocyanin was faster (FIG. 20C), which is likely because at pH 5 anthocyanin will be deprotonated in the internal phase. This deprotonation induces GMO's crystal structural change at the HIPE droplet interfaces and stabilizes as a bicontinuous cubic phase due to the electrostatic repulsion between negatively charged anthocyanin and the negatively charged headgroups of GMO (Negrini et al., “PH-Responsive Lyotropic Liquid Crystals for Controlled Drug Delivery,” Langmuir 27(9):5296-303 (2011), which is hereby incorporated by reference in its entirety). Such changes of the GMO crystal structure allows higher water absorptivity and thus a higher release rate (Negrini et al., “PH-Responsive Lyotropic Liquid Crystals for Controlled Drug Delivery,” Langmuir 27(9):5296-303 (2011); and Chang et al., “Effect of Dissolution Media and Additives on the Drug Release from Cubic Phase Delivery Systems,” J. Control. Release 46(3):215-22 (1997), which are hereby incorporated by reference in their entirety).

In addition to hydrophilic compounds, hydrophobic ones, such as β-carotene, can also be incorporated into the oleogel-structured external phase (FIGS. 20A, 21B). As shown in FIGS. 20D-20E, β-carotene released from the O-HIPE at a significantly higher rate (P<0.05) than the gel-in-gel HIPEs. The higher release might be due to the looser packing of the O-HIPE's internal phase. Thus the β-carotene in the external phase is more prone to diffuse out. However, β-carotene incorporated in the external phase of gel-in-gel HIPEs releases in a similarly slow fashion to those that are encapsulated in the 0/W Pickering HIPE (Tan et al., “Gelatin Particle-Stabilized High Internal Phase Emulsions as Nutraceutical Containers,” ACS Appl. Mater. Interfaces 6(16):13977-84 (2014), which is hereby incorporated by reference). These observations suggest the potential for such gel-in-gel HIPEs to be used as a long-term delivery system for both hydrophilic and hydrophobic compounds.

### Examples 4-8: Mitigating the Astringency of Acidified Whey Protein in Proteinaceous High Internal Phase Emulsion

### Materials and Methods for Examples 4-8

Materials. Fluorescein isothiocyanate isomers (≥90.0%), Nile red, sodium hydroxide (≥98.0%), and mucin from bovine submaxillary glands (BSM) were purchased from Sigma-Aldrich (St. Louis, Mo., USA). Hydrochloric acid (36.5-38%) was obtained from VWR International (Radnor, Pa., USA). Canola oil was purchased from a local supermarket (Ithaca, N.Y., USA). Kappa (κ), iota (ι), and lambda (λ)-carrageenan were provided by TIC Gums Incorporated (White Marsh, Md., USA). Glycerol monooleate (GMO; Capmul GMO-50 EP/NF) was kindly donated by Abitec Corporation (Columbus, Ohio, USA). Beeswax was kindly donated by Strahl & Pitsch, Inc. (West Babylon, N.Y., USA). WPI was kindly donated by Davisco Food International Inc. (Le Sueur, Minn., USA). All ingredients used for sensory studies are of food-grade.

Preparation of HIPEs.

W/O HIPEs were fabricated as described in Examples 1-2, with some modifications. In brief, the oil phase (1 wt % GMO, 3 wt % beeswax in canola oil) was heated to 70° C. Whey protein solutions of different concentration (25° C.) was added slowly to the heated oil mixture until the internal aqueous volume fractions reach 0.65, 0.70, 0.75, and 0.80, under constant high-shear homogenization at 10,000 rpm. The internal aqueous volume fraction was calculated as the volume of water phase included in the emulsion divided by the total volume of the emulsion. Half-way through the fabrication, the HIPEs were submerged in an ice bath to slowly cool down to 4° C.

Preparation of Whey Protein-Polysaccharide Complex (PPC).

PPCs were fabricated based on method reported by Wagoner et al., “Whey Protein—Pectin Soluble Complexes for Beverage Applications,” Food Hydrocolloids 63:130-8 (2017), which is hereby incorporated by reference in its entirety, with some modifications. 1 wt % whey protein solutions and 2 wt % polysaccharide solutions of carrageenans (κ-, ι-, λ-) were combined at ratio of 1:1 (v/v) at pH 6. The combined mixtures were then adjusted to pH 3.5 by slowly adding 0.1 v % phosphoric acid. Subsequently, the mixture was heated to 80° C. for 10 minutes. Further, PPCs solutions were concentrated to 20 wt % through heat evaporation.

Rheological Measurements of HIPEs.

The HIPEs were stored and measured at 25° C. The rheological measurements of the HIPEs were carried out through dynamic measurements on an AR 1000 Rheometer (TA instruments, New Castle, Del., USA), using a 40-mm plate geometry and a gap of 500 μm. The linear viscoelastic region was determined by a strain sweep at a frequency of 1 Hz from 0.0001 to 10. The G′ and G″ modulus were obtained through frequency sweeps from 0.1-10 rad/s using a fixed strain value of 0.0003.

Confocal Laser Scanning Microscopy.

The microstructure of HIPEs were inspected with a confocal laser scanning microscope (CLSM; LSM 710, Carl Zeiss, Gottingen, Germany). For CLSM, the internal and external phases were stained with fluorescein isothiocyanate isomers and Nile red at 1 mg/mL and 2 mg/mL, respectively. The HIPE structural morphology was studied using excitation/emission wavelengths of 488/515 nm and 492/518 nm, for fluorescein isothiocyanate isomers and Nile red, respectively.

Particle Size Measurement.

To measure the mean particle diameters of the droplets within the HIPEs samples, ImageJ software (v1.51) was utilized for image analysis. This instrument measures the mean particle size through calculation of the droplet diameter through image pixel analysis. At least 100 particles were analyzed for each sample.

Quartz Crystal Microbalance with Dissipation (QCM-D) Monitoring for Mucin-Protein Interactions.

The experiments were conducted using a Q-Sense Explorer single-module system (Biolin Scientific, Gothenburg, Sweden). First, a solution of 0.1 wt % of bovine submaxillary mucin in phosphate buffer at pH 7 (oral mucin model) was injected continuously to the QCM-D cell over the gold-coated SiO2 sensor (QSX 301) at flow rate of 0.3 mL/min at 25° C. After the mucin adsorbed on the sensor reached equilibrium, the coated sensor was washed with pH 7 phosphate buffer. This step generates the oral cavity conditions on the sensor (pH 7). Since the focus of this work is an acidified WPI at oral conditions, the pH of the injection cell is adjusted to pH 3.5 by injecting water (adjusted to pH 3.5 by 1 M HCl) into the QCM-D cell until the attached mucin reaches another equilibrium. 2nd layer of solution (0.2 wt % PPC (κ-, ι-, λ-)) was injected and was post-washed with buffer at pH 3.5. The 0.1 wt % WPI followed by 0.2 wt % carrageenan (κ-, ι-, λ) were sequentially injected as the 2nd layer on the mucin as a control.

All of the solutions were degassed. The frequencies and corresponding dissipations were measured simultaneously. The raw data was analyzed using the Composite Sauerbrey model on QCM-D DFind software (QScense) to quantify the mass of adsorbed molecules (Sauerbrey, “Schwingquarzen zur Wagung dünner Schichten and zur Mikrowägung,” Zeitschrift Für Physik 155(2):206-22 (1959), which is hereby incorporated by reference in its entirety). Each experiment was carried out in triplicate. To clean the sensors, sensors were heated in a cleaning solution (71 v % milli-Q water, 14.5 v % ammonia, and 14.5 v % hydrogen peroxide) at 75° C. for 5 min, rinsed with Milli-Q water, dried with nitrogen gas, and then UV-Ozone treated for 10 min.

Sensory Evaluation of HIPEs.

Volunteers of 15 adult males and females aged ≥18 years were recruited (Ithaca, N.Y., USA). The sensory evaluation studies were performed under the guidelines of human participants policy and standard operating procedures, and the experiments were approved by the ethics committee at the Cornell University (Protocol ID #1903008666). Informed consents were obtained from panelists of this study. The astringency of the protein-containing samples was evaluated in an untrained panel. Panelists were presented with two sets of samples, the protein-polysaccharide complex and proteinaceous HIPE samples. Each set compose of three different samples plus a control throughout the sensory test, with same concentrations of protein contents. Hedonic scale of 1-9 was used to describe the protein-polysaccharide complexes and HIPE samples by rating astringency, with 1 being least astringent, and 9 being extremely astringent. The whey protein solution (20 wt %, pH 3.5) sample was used as a control with a pre-set astringency score of 5. The samples were evaluated and compared to the control by the panelists. All of the samples were prepared a day before the evaluation and were stored in a refrigerated condition at 4° C. until samples were evaluated.

### Example 4—Proteinaceous HIPE

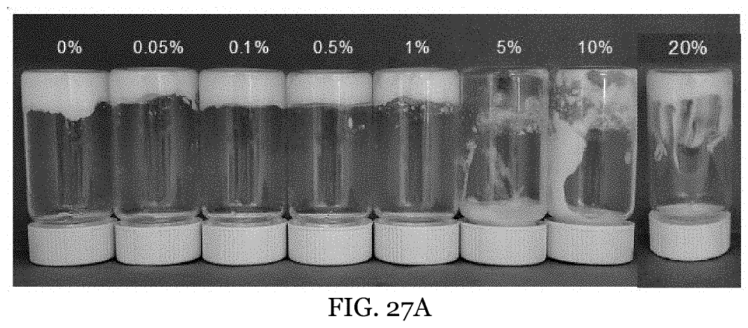

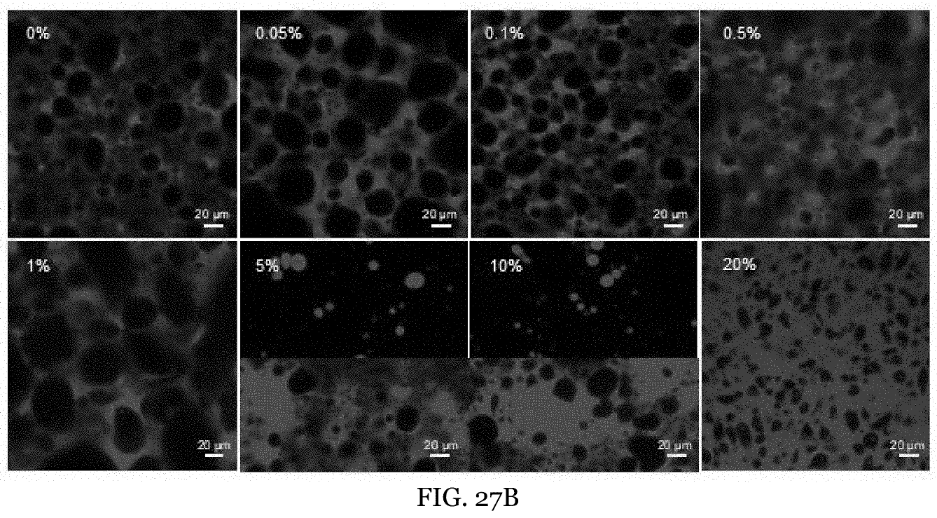

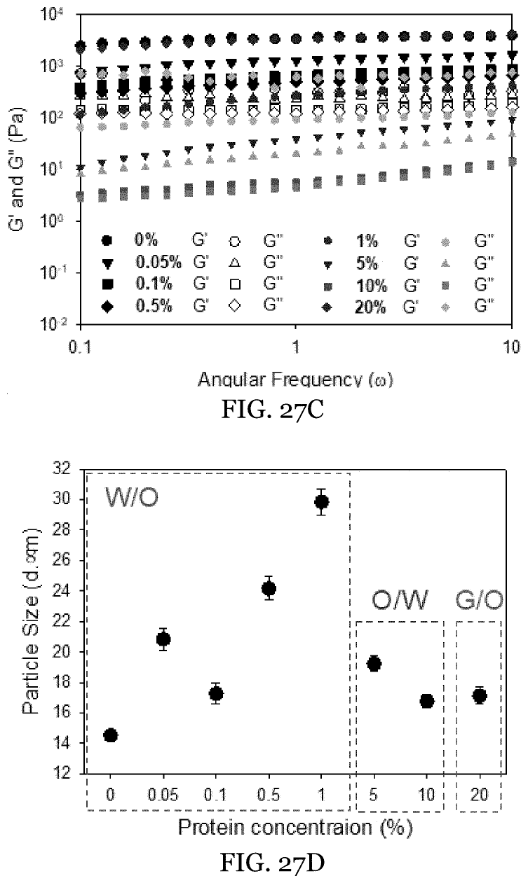

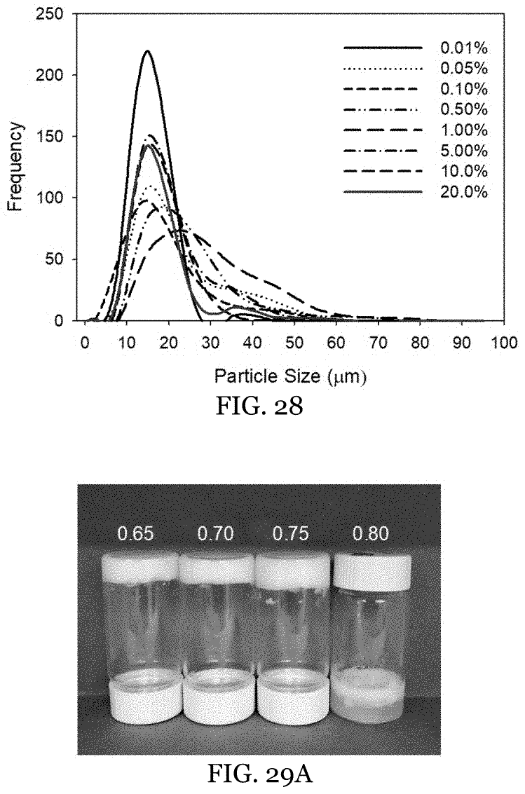

To mitigate protein astringency, the inventors proposed to encapsulate whey protein into the aqueous fraction of W/O HIPE. To provide a robust platform for protein mitigation, the loading capability of whey protein is very critical. Here, the maximum protein loading concentration was defined using the W/O HIPE formulation of Examples 1-2. Shown in FIG. 27A, the cutoff point of the highest protein loading concentration is 1%. When the microstructure of these HIPEs was investigated using confocal laser scanning microscopy (CLSM), phase separation occurred with 5% and 10% protein loading (FIG. 27B). This is due to the nature of WPI, which limits the loading capacity due to its high surface activity. In addition, at higher WPI concentration (20%) and pH of 3.5, WPI forms gel prior to HIPE fabrication (Alting et al., “Cold-Set Globular Protein Gels: Interactions, Structure and Rheology as a Function of Protein Concentration,” J. Agric. Food Chem. 51(10):3150-6 (2003), which is hereby incorporated by reference in its entirety). Consequently, HIPEs with 20% WPI cannot be formed, with unsuccessful packing of internal phase droplets, demonstrating the presence of gel-in-oil (G/O) emulsion. The rheological measurement indicated that the storage modulus (G′), decrease with increasing WPI loading (FIG. 27C). G′ is low when the phase separation occurs and again obtain its viscoelasticity for G/O emulsion. Besides rheological assessments, particle size of the internal droplets can also reflect the HIPE stability (FIG. 27D). Smaller droplet and narrower particle size distribution typically provide a higher emulsion stability. It was found that the droplet size and particle size distribution increase with increasing protein concentration up to 1%, which explains the decrease of G′ in rheological measurements (FIGS. 27C, 27D, 28; Table 6). These results suggest that 1% WPI is the maximum loading concentration, which is thus used in the subsequent measurements.

TABLE 6
Particle size of HIPEs made with different WPI concentrations in the aqueous phase.

| | WPI concentrations (%) | | | | | | | |
|---|---|---|---|---|---|---|---|---|
| | 0 | 0.05 | 0.10 | 0.50 | 1 | 5 | 10 | 20 |
| Particle Size | 14.53 ± 4.89 | 20.84 ± 10.79 | 17.26 ±9.85 | 24.17 ± 11.18 | 29.85 ± 12.6 | 19.22 ± 8.21 | 16.78 ± 5.92 | 17.12 ± 8.05 |
| (μm) | | | | | | | | |

### Example 5—The Optimal Packing Volume Fraction

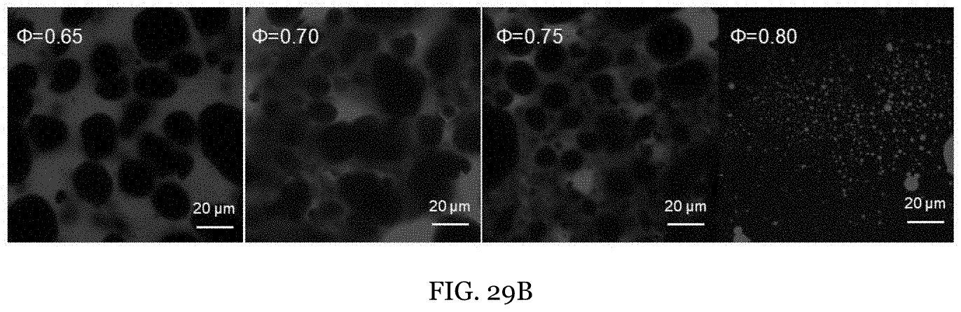

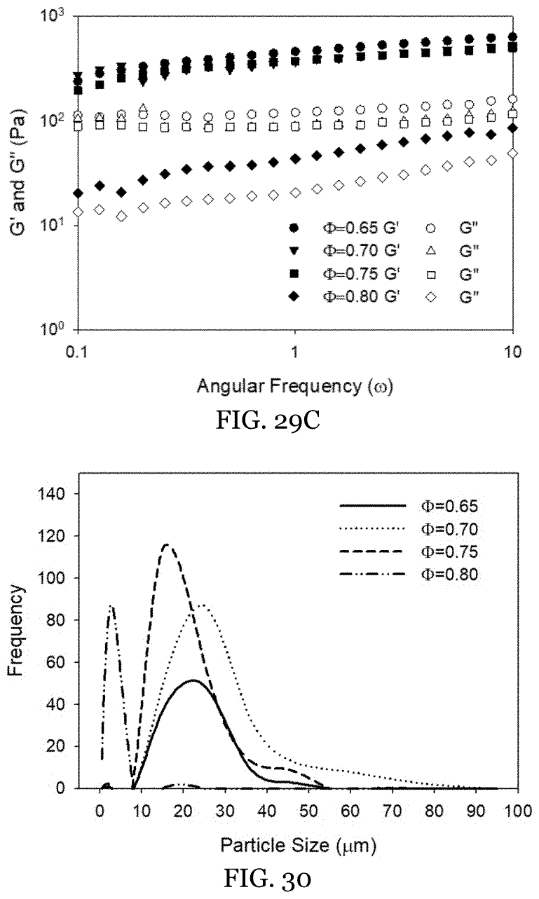

Next, the highest internal volume fractions (1) HIPE that can be reached with 1% WPI was investigated. As demonstrated in FIGS. 29A-29C, the highly concentrated emulsion can form viscoelastic HIPE with internal volume fractions of up to 0.75. When Φ=0.80, phase inversion occurs, thus forming an 01W emulsion. Considering the microstructure of HIPEs with different internal fractions, the particle sizes of the internal phase decreases with increasing 1, with a wider particle size distribution at Φ=0.65 and 0.70 (FIG. 29B; Table 7, FIG. 30). Additionally, rheological measurements show that HIPEs with Φ=0.65, 0.70 and 0.75 demonstrate the highest G′ value, indicating stronger viscoelasticity behavior (FIG. 29C). Therefore, Φ=0.75 was selected for all subsequent emulsions studied.

TABLE 7
Particle size of 1% WPI-HIPE made with
different internal phase volume ratios.

| | Internal phase volume ratio (Φ) | | | |
|---|---|---|---|---|
| | 0.65 | 0.70 | 0.75 | 0.80 |
| Particle Size | 23.01 ± 7.19 | 29.34 ± 13.52 | 20.67 ± 8.06 | 1.91 ± 1.2 |
| (μm) | | | | |

### Example 6—Stabilizing WPI Protein in the Internal Phase

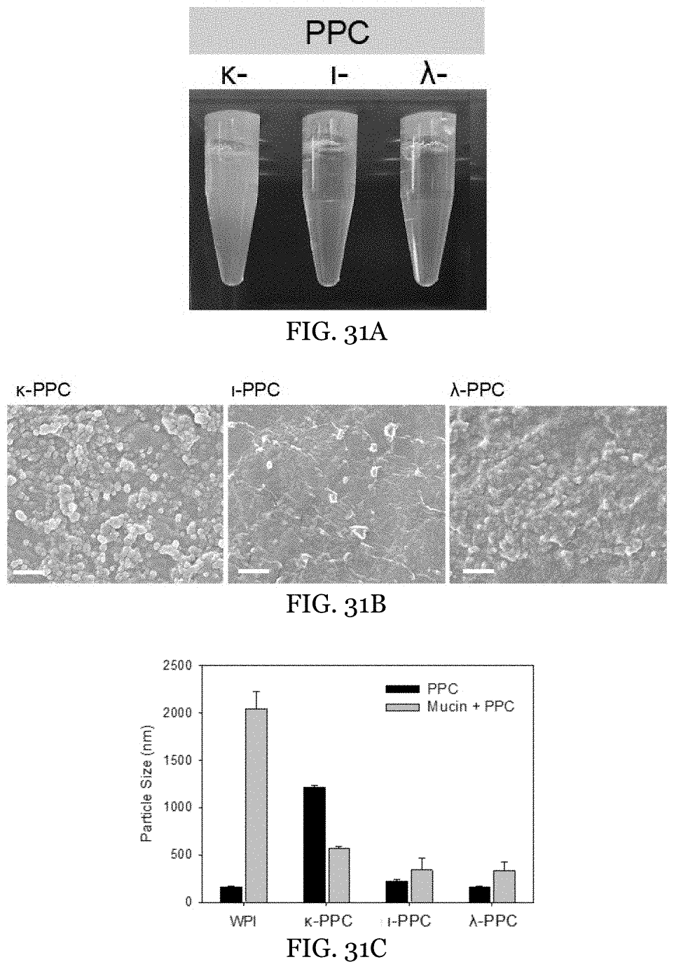

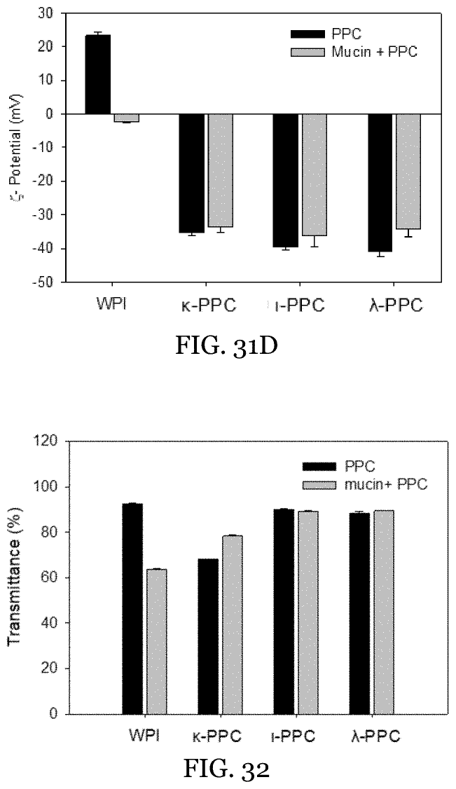

The WPI is surface active. Such surface activity can be further enhanced through heating above 80° C., due to the denaturation of WPI. Upon heat treatment, WPI exposes its hydrophobic site and becomes an efficient stabilizer for 01W emulsion. This property leads to technical difficulties in incorporating high concentrations of WPI into W/O HIPE, in which catastrophic phase inversion can occur readily. To overcome the high surface activity of WPI, while simultaneously incorporating high loads of WPI, the inventors propose to incorporate protein as protein-polysaccharide complexes (PPCs). Besides achieving high WPI loading into the W/O HIPE system, PPC can potentially play a role in mitigating protein astringency. Previously, Zeeb et al., “Modulation of the Bitterness of Pea and Potato Proteins by a Complex Coacervation Method, Food Funct. 9(4):2261-9 (2018), which is hereby incorporated by reference in its entirety, has reported that PPC can mitigate protein bitterness at acidic conditions. Currently, methods for mitigation of protein astringency focuses on decreasing protein interaction with saliva proteins. By the utilizing protein-polysaccharide complexation strategy, the inventors believe that not only the surface activity of WPI can be reduced, but the WPI-saliva protein interaction can also be minimized. To demonstrate, PPCs are formed based on the electrostatic interaction between two oppositely charged polymers. As the investigation was of WPI astringency at pH 3.5, which is below WPI's PI of 5.2, a negatively charged polysaccharide will be appropriate for PPC formation. Therefore, three negatively charged polysaccharides of carrageenans (κ-, ι-, λ-) were selected to form PPCs. However, upon introducing a positively charged protein and negatively charged polysaccharide, precipitation of PPCs occurred. This precipitation results in the undesirable alternation of the HIPE texture, and inhomogeneity of WPI dispersion. Previously reported by Wagoner et al., “Whey Protein—Pectin Soluble Complexes for Beverage Applications,” Food Hydrocolloids 63:130-8 (2017), which is hereby incorporated by reference in its entirety, a soluble PPC can be formed when protein and polysaccharide are mixed at charges of the same kind prior to the adjustments of pH, followed by a heating process to induce rearrangement of PPC. When the PPC is being heated, it self-rearranges into microgel featuring a WPI core and polysaccharide shell. Interestingly, in the presence of polysaccharide as outer layer of PPC, such PPC became soluble and did not precipitate out. Thus, this technique was applied to form soluble PPC in this system. FIGS. 31A-31D show that at a WPI to polysaccharide ratio of 1:2, soluble PPC can be formed with carrageenans (κ-, ι-, λ-) (FIG. 31A). Under scanning electron microscope (SEM) observation, the microstructures of the WPI-κ-carrageenan PPC (Λ-PPC), WPI-ι-carrageenan PPC, and WPI-λ-carrageenan (λ-PPC) show distinct complexes formation. Interestingly, κ-PPC aggregates into larger clumps, while ι-PPC and λ-PPC showed complexes that are well-dispersed (FIG. 31B). This is supported by the particle size measurement, where the particle size of κ-PPC is the largest, followed by ι-PPC and λ-PPC in the nanosized range (FIG. 31C). As the PPC are successfully formed and characterized, PPCs to mucin at pH 3.5 were further introduced, simulating oral conditions where PPCs interacts with saliva protein (mucin). The interaction of WPI and PPCs with mucin can be briefly demonstrated with particle size and ζ-potential measurements. Expectedly, WPI-mucin are shown to form large complexes at micron range (2.04±0.18 μm), which is also reflected in the increased solution turbidity (FIGS. 31C, 32). This large complex formation can be attributed to the strong electrostatic interaction between the positively charged WPI and negatively charged mucin (FIG. 31D). Interestingly, all PPCs (κ, ι-, λ-) formed relatively small complexes with mucin, and are strongly negatively charged (FIGS. 31C, 31D). Such results indicate that PPCs and mucin are electrostatically repelling, suggesting less PPCs and mucin interaction at pH 3.5.

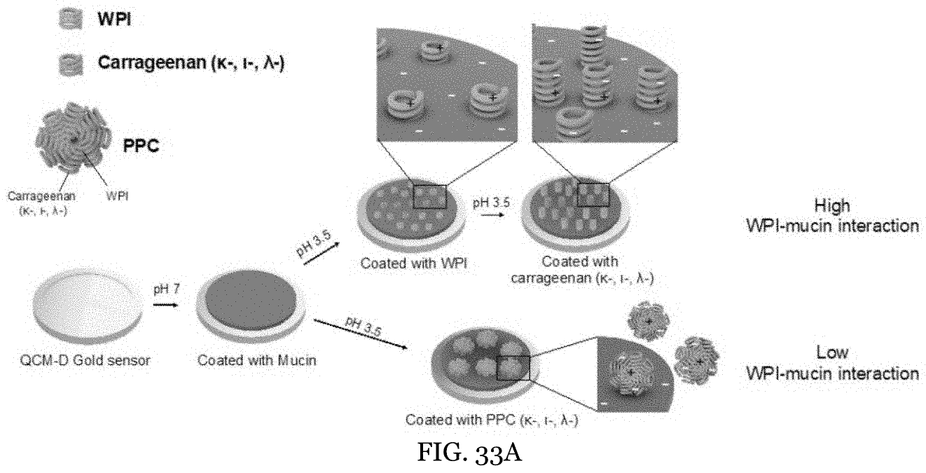

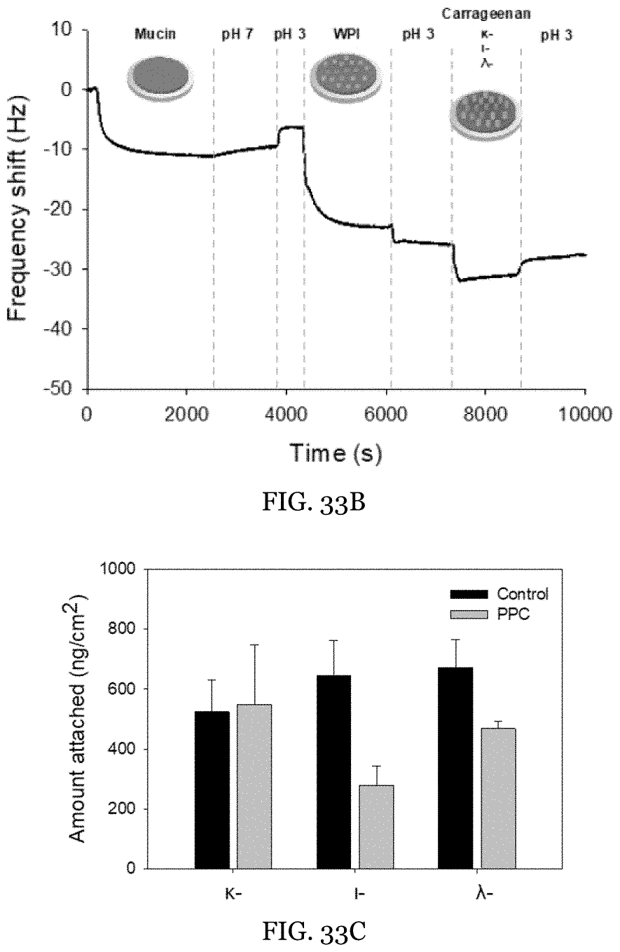

To further understand the proposed mechanism, experiments of quartz crystal microbalance with dissipation monitoring (QCM-D) simulating the interaction of acidified WPI and PPCs (κ-, ι-, λ-) with saliva mucin at oral conditions (pH 7) were conducted. Briefly, QCM-D monitors the variation of frequency (4f) and dissipation (4D) of the sensor when external mass (molecular interaction) is added. This can be useful in identifying interaction strength between two compounds (Hook et al., “The QCM-D Technique for Probing Biomacromolecular Recognition Reactions,” Piezoelectric Sensors Berlin, Heidelberg: Springer, pp. 425-47 (2013); Marchuk et al., “Mechanistic Investigation via QCM-D into the Color Stability Imparted to Betacyanins by the Presence of Food Grade Anionic Polysaccharides,” Food Hydrocolloids 93:226-34 (2019); and Voinova et al., “Viscoelastic Acoustic Response of Layered Polymer Films at Fluid-Solid Interfaces: Continuum Mechanics Approach,” Physica Scripta 59(5):391-6 (1999), which are hereby incorporated by reference in their entirety). The experiment was usually done by sequentially depositing layers of components onto the sensors, alternating with a series of buffer wash to reach equilibrium, and quantify the components remained on the sensors. To understand the reduced interaction of oral mucin with WPI by forming PPC, a layer of negatively charged mucin was coated onto the QCM-D gold sensor at pH 7, and introduced either acidified (pH 3.5) WPI or PPCs to monitor the gold sensor's frequency and dissipation change, the process is shown in FIG. 33A. As a control, the WPI was firstly attached onto negatively charged mucin, followed by attachment of carrageenans (FIG. 33B). The quantitative attachment of the WPI-carrageenan mixture and PPCs onto mucin layer can be calculated through a Composite Sauerbrey model (FIG. 33C) (Sauerbrey, “Schwingquarzen zur Wagung dünner Schichten and zur Mikrowägung,” Zeitschrift Für Physik 155(2):206-22 (1959), which is hereby incorporated by reference in its entirety). As shown in FIG. 33C, κ-PPC and WPI-κ-carrageenan (control) attached similarly to mucin (P>0.05), and this might be due to the aggregation and larger particle sizes of κ-PPC (FIG. 31C). Besides κ-PPC, ι-PPC and λ-PPC both showed significantly reduced mucin interaction (P<0.05) compared with the controls and are concordant with the particle size results from FIG. 31C. The particle size and QCM-D results suggest that ι-PPC and λ-PPC can be an effective way to minimize WPI-mucin interactions thus lowering protein astringency.

### Example 7—Incorporation of PPC into HIPEs

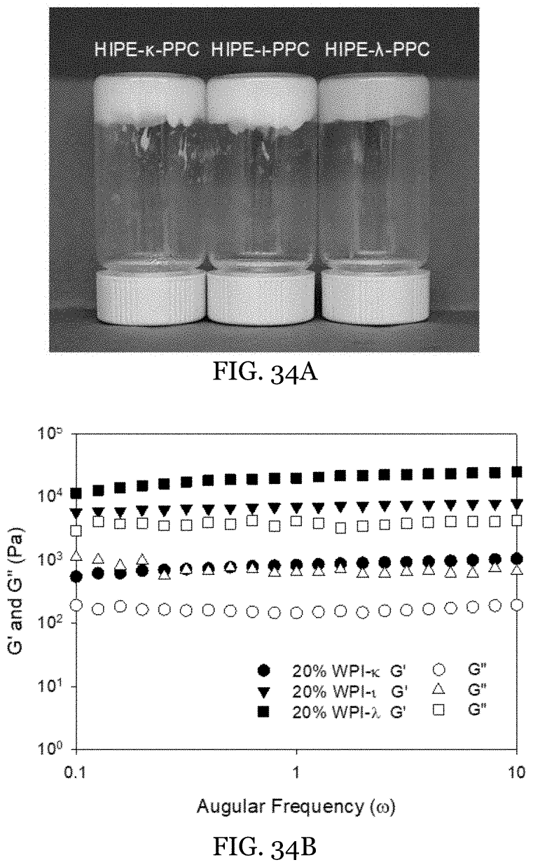

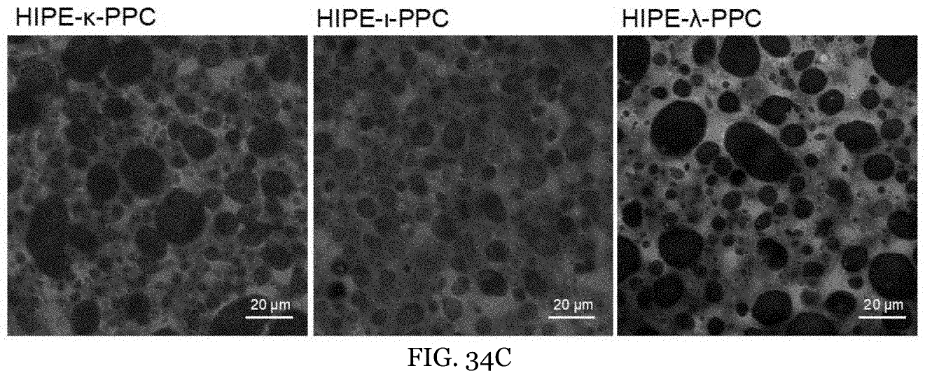

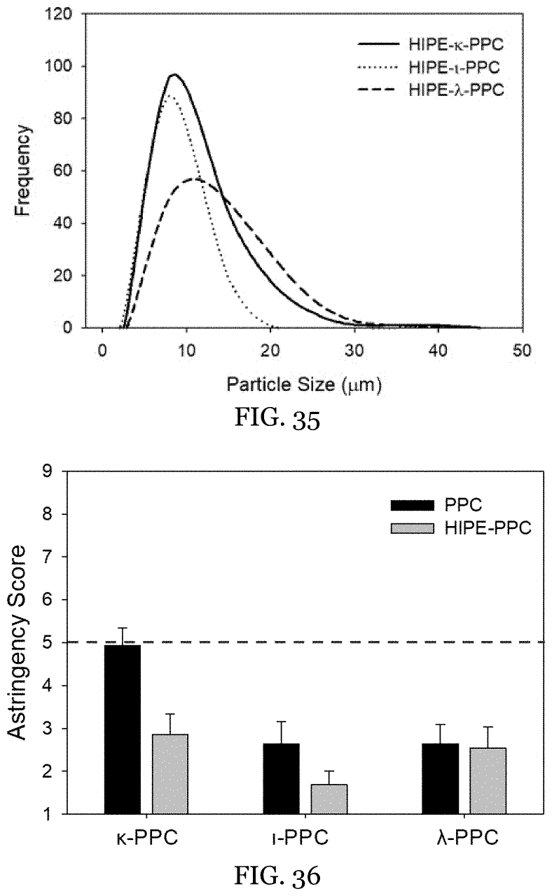

Due to the promising solubility characteristic of PPC, as well as providing less WPI-mucin interaction, it was decided to incorporate PPC as the internal phase for HIPE formation. As mentioned previously, WPI can only be successfully loaded into W/O HIPE up to 1% concentration (FIG. 27A). Surprisingly, when loading WPI as PPCs into the HIPE, protein loading as high as 20% WPI can be achieved (FIG. 34A). The rheological measurement shows that with 20% WPI loading, ι-PPC and λ-PPC lead to higher G′ of HIPE than κ-PPC (FIG. 34B). Compared to HIPEs formed with only 1% WPI (FIG. 27C), the PPC incorporation confers HIPEs with higher viscoelastic properties (FIG. 34B). However, the particle size of HIPEs made with λ-PPC is larger than those with κ-PPC and ι-PPC, simultaneously showing a wider particle size dispersion (FIGS. 34C, 35; Table 8). As highlighted above, larger particle size would cause instability of HIPEs.

TABLE 8
Particle size of HIPEs incorporating 20% of κ-, ι-, λ-PPC.

| | PPC type | | |
|---|---|---|---|
| | κ-PPC | ι-PPC | λ-PPC |
| Particle Size | 9.65 ± 5.06 | 7.97 ± 2.55 | 12.13 ± 5.65 |
| (μm) | | | |

### Example 8—Sensory Evaluation of the Astringency of PPC

The potential astringency mitigating effect by understanding WPI-mucin interactions were demonstrated. However, these are limited to in vitro observations. To directly evaluate the astringency mitigation by PPC, a sensory study on PPC ι-, containing 10% WPI was carried out. Meanwhile, 10% WPI solution was used as a control (shown as a dashed line, FIG. 36). Interestingly, ι-PPC and λ-PPC showed significant decreases in astringency intensity while κ-PPC did not as compared to the control (FIG. 36). This was supported by the QCM-D measurement where κ-PPC interacts with mucin more and results in astringent tastes (FIG. 33C). When the PPCs are further incorporated into HIPEs, the WPI astringency perception are largely reduced (FIG. 36). Among all HIPE-PPC evaluation, HIPE-ι-PPC showed the lowest astringent taste, while HIPE-κ-PPC and HIPE-λ-PPC were shown to be similarly astringent. The results demonstrate the potential of using HIPE as an effective encapsulation method to reduce protein astringency.

### Example 9—Probiotics in HIPEs

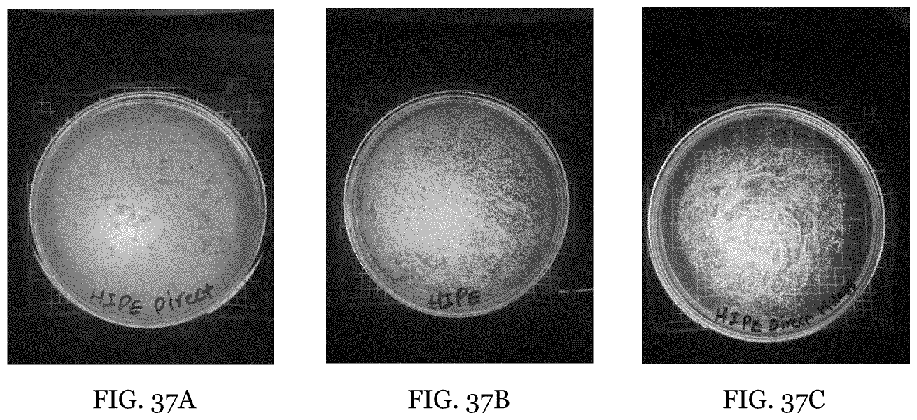

Using procedures similar to those set forth above, W/O HIPEs incorporating the probiotic Lactobacillus rhamnosus probiotic can be prepared. Here, the probiotic was incorporated into an O-HIPE with canola oil as an oleaginous carrier, GMO as a surfactant, and beeswax as an external structurant. The HIPEs were tested for maintenance of probiotic viability when stored at 25° C. by testing for growth of the probiotic after HIPEs were plated on De Man, Rogosa and Sharpe (MRS) agar growth medium and incubated at 37° C. The probiotic was found to be viable for at least 14 days (FIGS. 37A-37C).

### Example 10—Application of HIPEs as Butter, Food Spread, and Proteinaceous Spreadable Products

HIPEs (O-HIPE & Gel-in-gel HIPEs) incorporating various protein sources can be prepared using procedures similar to those set forth above.

Milk Protein Concentrate.

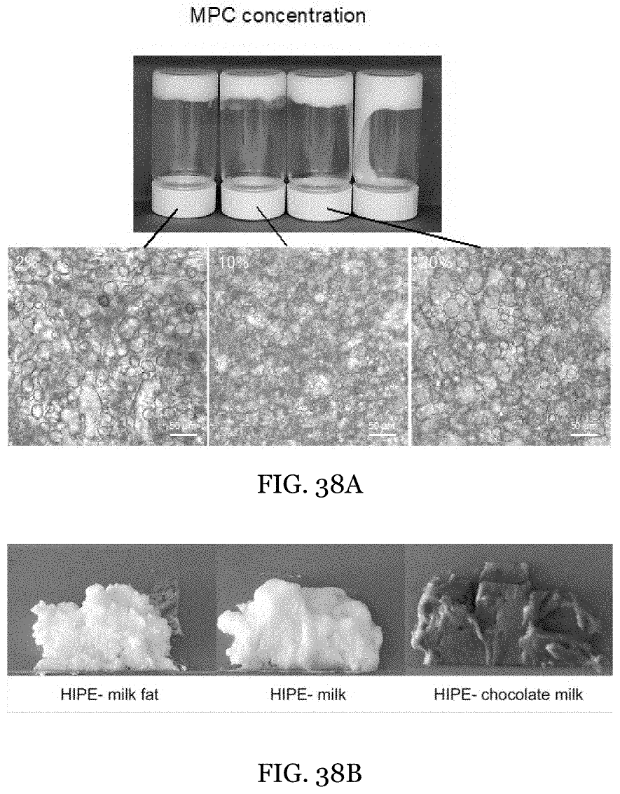

Using procedures similar to those set forth above, milk protein concentrate (MPC) has been loaded, at a level of up to 20 wt %, into the HIPE internal phase (FIG. 38A).

Milk Fats.

In addition, 0=0.68-0.74 W/O HIPEs have been formed using other materials, such as milk fats (Ghee), without the addition of any other surfactants or structurants (e.g. beeswax or carrageenans). Here, the milk fats are used as an oleogel; the liquid oil portion corresponds to the oleaginous carrier, surfactants are those naturally found in ghee, and the solid fats corresponds to the external structurant. Milk fat contains fat crystals and natural surfactants that can be used to make HIPEs. The internal phase can be successfully replaced by either milk, chocolate milk (FIG. 38B), or other aqueous juices.

By modifying the materials of the external phases and internal phases, these HIPEs can be used as spreadable food products, such as low-fat mayonnaise and butter products (e.g. butter substitutes, low-calorie butters, and food butter alternatives). They can also incorporate other internal materials suitable for spreadable food products.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A water-in-oil emulsion comprising:
an aqueous internal phase; and
an oleogel external phase comprising:
an oleaginous carrier;
a surfactant; and
an external structurant.

2. The water-in-oil emulsion of claim 1, wherein said aqueous internal phase comprises at least 80 wt % water.

3. The water-in-oil emulsion of claim 1, wherein said oleaginous carrier is selected from the group consisting of almond oil, apricot kernel oil, argan oil, avocado oil, baobab oil, camelina oil, canola oil, carrot oil, castor oil, chile oil, citronella oil, corn oil, cottonseed oil, cranberry seed oil, flax seed oil, grapeseed oil, hazelnut oil, hemp seed oil, jojoba oil, macadamia nut oil, meadowfoam seed oil, mustard oil, oat emollient, olive oil, peanut oil, pine seed oil, poppy seed oil, rapeseed oil, red raspberry seed oil, rice bran oil, rose hip oil, safflower oil, sesame oil, sesame seed oil, soybean oil, sunflower oil, tea oil, truffle oil, walnut oil, wheat germ oil, fish oil, and combinations thereof.

4. The water-in-oil emulsion of claim 1, wherein said oleogel external phase comprises at least 80 wt % of the oleaginous carrier.

5. The water-in-oil emulsion of claim 1, wherein said surfactant is selected from the group consisting of ethoxylated linear alcohols, ethoxylated alkyl phenols, ethoxylated thiols, acid ethoxylated fatty acids, glycerol esters, esters of hexitols and cyclic anhydrohexitols, amine and amide derivatives, alkylpolyglucosides, ethleneoxide/propyleneoxide copolymers, polyalcohols and ethyoxylated polyalcohols, thiols (mercaptans) and derivatives, and combinations thereof.

6. The water-in-oil emulsion of claim 1, wherein said surfactant is present in an amount from 1 wt % to 3 wt % of the oleogel external phase.

7. The water-in-oil emulsion of claim 1, wherein said external structurant is selected from the group consisting of cocoa butter, coconut oil, margarine, palm kernel oil, palm oil, beef fat, beeswax, butter, chicken fat, ghee, milk fat, pork fat, hydrogenated oils, partially hydrogenated oils, and combinations thereof.

8. The water-in-oil emulsion of claim 1, wherein said external structurant is present in an amount from 1 wt % to 20 wt % of the oleogel external phase.

9. The water-in-oil emulsion of claim 1, wherein the water-in-oil emulsion has an aqueous internal phase volume fraction (Φ) of from 0.65 to 0.80.

10. The water-in-oil emulsion of claim 1 further comprising:
interfacial Pickering crystals on surfaces of aqueous phase droplets within the water-in-oil emulsion.

11. The water-in-oil emulsion of claim 1, wherein the water-in-oil emulsion includes aqueous phase droplets having a mean particle diameter of 10 μm to 30 μm.

12. The water-in-oil emulsion of claim 1, wherein the water-in-oil emulsion is stable at 25° C. for at least two days.

13. A water-in-oil emulsion, comprising:
a hydrogel aqueous internal phase comprising:
an internal structurant; and
an oleogel external phase comprising:
an oleaginous carrier;
surfactant; and
an external structurant.

14. The water-in-oil emulsion of claim 13, wherein said aqueous internal phase comprises at least 80 wt % water.

15. The water-in-oil emulsion of claim 13, wherein said oleaginous carrier is selected from the group consisting of almond oil, apricot kernel oil, argan oil, avocado oil, baobab oil, camelina oil, canola oil, carrot oil, castor oil, chile oil, citronella oil, corn oil, cottonseed oil, cranberry seed oil, flax seed oil, grapeseed oil, hazelnut oil, hemp seed oil, jojoba oil, macadamia nut oil, meadowfoam seed oil, mustard oil, oat emollient, olive oil, peanut oil, pine seed oil, poppy seed oil, rapeseed oil, red raspberry seed oil, rice bran oil, rose hip oil, safflower oil, sesame oil, sesame seed oil, soybean oil, sunflower oil, tea oil, truffle oil, walnut oil, wheat germ oil, fish oil, and combinations thereof.

16. The water-in-oil emulsion of claim 13, wherein said oleogel external phase comprises at least 80 wt % of the oleaginous carrier.

17. The water-in-oil emulsion of claim 13, wherein said surfactant is selected from the group consisting of ethoxylated linear alcohols, ethoxylated alkyl phenols, ethoxylated thiols, acid ethoxylated fatty acids, glycerol esters, esters of hexitols and cyclic anhydrohexitols, amine and amide derivatives, alkylpolyglucosides, ethleneoxide/propyleneoxide copolymers, polyalcohols and ethyoxylated polyalcohols, thiols (mercaptans) and derivatives, and combinations thereof.

18. The water-in-oil emulsion of claim 13, wherein said surfactant is present in an amount from 1 wt % to 3 wt % of the oleogel external phase.

19. The water-in-oil emulsion of claim 13, wherein said external structurant is selected from the group consisting of coca butter, coconut oil, margarine, palm kernel oil, palm oil, beef fat, beeswax, butter, chicken fat, ghee, milk fat, pork fat, hydrogenated oils, partially hydrogenated oils, and combinations thereof.

20. The water-in-oil emulsion of claim 13, wherein said external structurant is present in an amount from 1 wt % to 20 wt % of the oleogel external phase.

21. The water-in-oil emulsion of claim 13, wherein the water-in-oil emulsion has an aqueous internal phase volume fraction (Φ) of from 0.65 to 0.80.

22. The water-in-oil emulsion of claim 13 further comprising:
interfacial Pickering crystals on surfaces of aqueous phase droplets within the water-in-oil emulsion.

23. The water-in-oil emulsion of claim 13, wherein the water-in-oil emulsion includes droplets having a mean particle diameter of 10 μm to 30 μm.

24. The water-in-oil emulsion of claim 13, wherein the water-in-oil emulsion is stable at 25° C. for at least two days.

25. The water-in-oil emulsion of claim 13, wherein said internal structurant is selected from the group consisting of: proteins, polysaccharides, biosynthetic polypeptides, oligopeptides, PEGylated polymers, and combinations thereof.

26. The water-in-oil emulsion of claim 13, wherein said internal structurant is present in an amount from 0.5 wt % to 2 wt % of the hydrogel aqueous internal phase.

27-46. (canceled)
